(19) United States
(12) Patent Application Publication
(10) Pub. No.: US 2017/0238859 A1
(43) Pub. Date: Aug. 24, 2017

(54) MENTAL STATE DATA TAGGING AND MOOD ANALYSIS FOR DATA COLLECTED FROM MULTIPLE SOURCES

(71) Applicant: Affectiva, Inc., Boston, MA (US)

(72) Inventors: Richard Scott Sadowsky, Sturbridge, MA (US); Rana el Kaliouby, Milton, MA (US)

(73) Assignee: Affectiva, Inc., Boston, MA (US)

(21) Appl. No.: 15/589,399

(22) Filed: May 8, 2017

Related U.S. Application Data
(63) Continuation-in-part of application No. 14/214,704, filed on Mar. 15, 2014, now Pat. No. 9,646,046.
Application No. 14/214,704 is a continuation-in-part of application No. 14/144,413, filed on Dec. 30, 2013.
Application No. 14/144,413 is a continuation-in-part of application No. 13/153,745, filed on Jun. 6, 2011, now abandoned.
Application No. 13/153,745 is a continuation-in-part of application No. 14/064,136, filed on Oct. 26, 2013, now Pat. No. 9,204,836.
Application No. 14/064,136 is a continuation-in-part of application No. 15/382,087, filed on Dec. 16, 2016.
Application No. 15/382,087 is a continuation-in-part of application No. 13/153,745, filed on Jun. 6, 2011, now abandoned.
Application No. 15/382,087 is a continuation-in-part of application No. 15/262,197, filed on Sep. 12, 2016.
Application No. 15/262,197 is a continuation-in-part of application No. 14/796,419, filed on Jul. 10, 2015.
Application No. 14/796,419 is a continuation-in-part of application No. 13/153,745, filed on Jun. 6, 2011, now abandoned.
Application No. 14/796,419 is a continuation-in-part of application No. 14/460,915, filed on Aug. 15, 2014.
Application No. 14/460,915 is a continuation-in-part of application No. 13/153,745, filed on Jun. 6, 2011, now abandoned.
(60) Provisional application No. 62/370,421, filed on Aug. 3, 2016.
(60) Provisional application No. 62/469,591, filed on Mar. 10, 2017.
(60) Provisional application No. 61/790,461, filed on Mar. 15, 2013.
(60) Provisional application No. 61/793,761, filed on Mar. 15, 2013.
(60) Provisional application No. 61/789,038, filed on Mar. 15, 2013.
(60) Provisional application No. 61/798,731, filed on Mar. 15, 2013.
(60) Provisional application No. 61/844,478, filed on Jul. 10, 2013.
(60) Provisional application No. 61/916,190, filed on Dec. 14, 2013.
(60) Provisional application No. 61/924,252, filed on Jan. 7, 2014.
(60) Provisional application No. 61/927,481, filed on Jan. 15, 2014.
(60) Provisional application No. 61/747,651, filed on Dec. 31, 2012.
(60) Provisional application No. 61/747,810, filed on Dec. 31, 2012.
(60) Provisional application No. 61/793,761, filed on Mar. 15, 2013.
(60) Provisional application No. 61/790,461, filed on Mar. 15, 2013.
(60) Provisional application No. 61/789,038, filed on Mar. 15, 2013.
(60) Provisional application No. 61/798,731, filed on Mar. 15, 2013.
(60) Provisional application No. 61/844,478, filed on Jul. 10, 2013.
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.
(60) Provisional application No. 61/719,383, filed on Oct. 27, 2012.
(60) Provisional application No. 61/747,651, filed on Dec. 31, 2012.
(60) Provisional application No. 61/747,810, filed on Dec. 31, 2012.
(60) Provisional application No. 61/793,761, filed on Mar. 15, 2013.
(60) Provisional application No. 61/790,461, filed on Mar. 15, 2013.
(60) Provisional application No. 61/789,038, filed on Mar. 15, 2013.
(60) Provisional application No. 61/798,731, filed on Mar. 15, 2013.
(60) Provisional application No. 61/844,478, filed on Jul. 10, 2013.
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.
(60) Provisional application No. 62/217,872, filed on Sep. 12, 2015.
(60) Provisional application No. 62/222,518, filed on Sep. 23, 2015.
(60) Provisional application No. 62/265,937, filed on Dec. 10, 2015.
(60) Provisional application No. 62/273,896, filed on Dec. 31, 2015.
(60) Provisional application No. 62/301,558, filed on Feb. 29, 2016.
(60) Provisional application No. 62/370,421, filed on Aug. 3, 2016.
(60) Provisional application No. 62/023,800, filed on Jul. 11, 2014.
(60) Provisional application No. 62/047,508, filed on Sep. 8, 2014.
(60) Provisional application No. 62/082,579, filed on Nov. 20, 2014.
(60) Provisional application No. 62/128,974, filed on Mar. 5, 2015.
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.
(60) Provisional application No. 61/867,007, filed on Aug. 16, 2013.
(60) Provisional application No. 61/924,252, filed on Jan. 7, 2014.
(60) Provisional application No. 61/916,190, filed on Dec. 14, 2013.
(60) Provisional application No. 61/927,481, filed on Jan. 15, 2014.
(60) Provisional application No. 61/953,878, filed on Mar. 16, 2014.
(60) Provisional application No. 61/972,314, filed on Mar. 30, 2014.
(60) Provisional application No. 62/023,800, filed on Jul. 11, 2014.
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.

(51) Int. Cl. A61B 5/16; A61B 5/0205; A61B 5/1171; A61B 5/00

(52) U.S. Cl. CPC A61B 5/165; A61B 5/0077; A61B 5/7278; A61B 5/0022; A61B 5/02055; A61B 5/6801; A61B 5/7267; A61B 5/1176; A61B 2576/00

(57) ABSTRACT

Mental state data useful for determining mental state information on an individual, such as video of an individual's face, is captured. Additional data that is helpful in determining the mental state information, such as contextual information, is also determined. Intermittent mental state data is interpolated. The data and additional data allow interpretation of individual mental state information. The additional data is tagged to the mental state data. At least some of the mental state data, along with the tagged data, is analyzed to produce further mental state information. A mood measurement is a result of the analysis.

### RELATED APPLICATIONS

This application claims the benefit of U.S. provisional patent applications “Deep Convolutional Neural Network Analysis of Images for Mental States” Ser. No. 62/370,421, filed Aug. 3, 2016 and “Image Analysis for Two-Sided Data Hub” Ser. No. 62/469,591, filed Mar. 10, 2017. This application is also a continuation-in-part of U.S. patent application “Mental State Data Tagging for Data Collected from Multiple Sources” Ser. No. 14/214,704, filed Mar. 15, 2014, which claims the benefit of U.S. provisional patent applications “Mental State Data Tagging for Data Collected from Multiple Sources” Ser. No. 61/790,461, filed Mar. 15, 2013, “Mental State Analysis Using Heart Rate Collection Based on Video Imagery” Ser. No. 61/793,761, filed Mar. 15, 2013, “Mental State Analysis Using Blink Rate” Ser. No. 61/789,038, filed Mar. 15, 2013, “Mental State Well Being Monitoring” Ser. No. 61/798,731, filed Mar. 15, 2013, “Personal Emotional Profile Generation” Ser. No. 61/844,478, filed Jul. 10, 2013, “Heart Rate Variability Evaluation for Mental State Analysis” Ser. No. 61/916,190, filed Dec. 14, 2013, “Mental State Analysis Using an Application Programming Interface” Ser. No. 61/924,252, filed Jan. 7, 2014, and “Mental State Analysis for Norm Generation” Ser. No. 61/927,481, filed Jan. 15, 2014.

This application is also a continuation-in-part of U.S. patent application “Collection of Affect Data from Multiple Mobile Devices” Ser. No. 14/144,413, filed Dec. 30, 2013, which claims the benefit of U.S. provisional patent applications “Optimizing Media Based on Mental State Analysis” Ser. No. 61/747,651, filed Dec. 31, 2012, “Collection of Affect Data from Multiple Mobile Devices” Ser. No. 61/747,810, filed Dec. 31, 2012, “Mental State Analysis Using Heart Rate Collection Based on Video Imagery” Ser. No. 61/793,761, filed Mar. 15, 2013, “Mental State Data Tagging for Data Collected from Multiple Sources” Ser. No. 61/790,461, filed Mar. 15, 2013, “Mental State Analysis Using Blink Rate” Ser. No. 61/789,038, filed Mar. 15, 2013, “Mental State Well Being Monitoring” Ser. No. 61/798,731, filed Mar. 15, 2013, and “Personal Emotional Profile Generation” Ser. No. 61/844,478, filed Jul. 10, 2013.

This application is also a continuation-in-part of U.S. patent application “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Data Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011.

This application is also a continuation-in-part of U.S. patent application “Sporadic Collection of Mobile Affect Data” Ser. No. 14/064,136, filed Oct. 26, 2013, which claims the benefit of U.S. provisional patent applications “Sporadic Collection of Affect Data” Ser. No. 61/719,383, filed Oct. 27, 2012, “Optimizing Media Based on Mental State Analysis” Ser. No. 61/747,651, filed Dec. 31, 2012, “Collection of Affect Data from Multiple Mobile Devices” Ser. No. 61/747,810, filed Dec. 31, 2012, “Mental State Analysis Using Heart Rate Collection Based on Video Imagery” Ser. No. 61/793,761, filed Mar. 15, 2013, “Mental State Data Tagging for Data Collected from Multiple Sources” Ser. No. 61/790,461, filed Mar. 15, 2013, “Mental State Analysis Using Blink Rate” Ser. No. 61/789,038, filed Mar. 15, 2013, “Mental State Well Being Monitoring” Ser. No. 61/798,731, filed Mar. 15, 2013, and “Personal Emotional Profile Generation” Ser. No. 61/844,478, filed Jul. 10, 2013.

This application is also a continuation-in-part of U.S. patent application “Mental State Analysis Using Web Servers” Ser. No. 15/382,087, filed Dec. 17, 2016, which is a continuation in part of “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Data Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011.

The patent application “Mental State Analysis Using Web Servers” Ser. No. 15/382,087, filed Dec. 17, 2016, is also a continuation-in-part of U.S. patent application “Mental State Event Signature Usage” Ser. No. 15/262,197, filed Sep. 12, 2016, which claims the benefit of U.S. provisional patent applications “Mental State Event Signature Usage” Ser. No. 62/217,872, filed Sep. 12, 2015, “Image Analysis In Support of Robotic Manipulation” Ser. No. 62/222,518, filed Sep. 23, 2015, “Analysis of Image Content with Associated Manipulation of Expression Presentation” Ser. No. 62/265,937, filed Dec. 10, 2015, “Image Analysis Using Sub-Sectional Component Evaluation To Augment Classifier Usage” Ser. No. 62/273,896, filed Dec. 31, 2015, “Analytics for Live Streaming Based on Image Analysis within a Shared Digital Environment” Ser. No. 62/301,558, filed Feb. 29, 2016, and “Deep Convolutional Neural Network Analysis of Images for Mental States” Ser. No. 62/370,421, filed Aug. 3, 2016.

The patent application “Mental State Event Signature Usage” Ser. No. 15/262,197, filed Sep. 12, 2016, is also a continuation-in-part of U.S. patent application “Mental State Event Definition Generation” Ser. No. 14/796,419, filed Jul. 10, 2015, which claims the benefit of U.S. provisional patent applications “Mental State Event Definition Generation” Ser. No. 62/023,800, filed Jul. 11, 2014, “Facial Tracking with Classifiers” Ser. No. 62/047,508, filed Sep. 8, 2014, “Semiconductor Based Mental State Analysis” Ser. No. 62/082,579, filed Nov. 20, 2014, and “Viewership Analysis Based On Facial Evaluation” Ser. No. 62/128,974, filed Mar. 5, 2015.

The patent application “Mental State Event Definition Generation” Ser. No. 14/796,419, filed Jul. 10, 2015 is also a continuation-in-part of U.S. patent application “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011.

The patent application “Mental State Event Definition Generation” Ser. No. 14/796,419, filed Jul. 10, 2015 is also a continuation-in-part of U.S. patent application “Mental State Analysis Using an Application Programming Interface” Ser. No. 14/460,915, Aug. 15, 2014, which claims the benefit of U.S. provisional patent applications “Application Programming Interface for Mental State Analysis” Ser. No. 61/867,007, filed Aug. 16, 2013, “Mental State Analysis Using an Application Programming Interface” Ser. No. 61/924,252, filed Jan. 7, 2014, “Heart Rate Variability Evaluation for Mental State Analysis” Ser. No. 61/916,190, filed Dec. 14, 2013, “Mental State Analysis for Norm Generation” Ser. No. 61/927,481, filed Jan. 15, 2014, “Expression Analysis in Response to Mental State Express Request” Ser. No. 61/953,878, filed Mar. 16, 2014, “Background Analysis of Mental State Expressions” Ser. No. 61/972,314, filed Mar. 30, 2014, and “Mental State Event Definition Generation” Ser. No. 62/023,800, filed Jul. 11, 2014.

The patent application “Mental State Analysis Using an Application Programming Interface” Ser. No. 14/460,915, Aug. 15, 2014 is also a continuation-in-part of U.S. patent application “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011.

The foregoing applications are each hereby incorporated by reference in their entirety.

### FIELD OF ART

This application relates generally to the analysis of mental states and more particularly to the tagging and mood analysis of mental state data collected from multiple sources.

### BACKGROUND

People spend an ever-increasing amount of time interacting with computers, and consume a vast amount of computer-delivered media. This interaction can be for many different reasons, such as a desire to find educational or entertaining content, to interact with others using social media, to create documents, and to play games, to name a few examples.

In some cases, the human-computer interaction can take the form of a person performing a task using a software-based tool running on a computer. Examples include creating a document, editing a video, and/or doing one or more of the numerous other activities a modern computer can perform. The person might find the execution of certain activities interesting or even exciting, and might be surprised at how easy it is to perform the activity on their computer. The person can become excited, happy, or content as he or she performs an interesting or exciting activity. On the other hand, the person might find some activities difficult to perform, and might become frustrated or even angry with the computer or software tool. In some cases, for example, users are surveyed in an attempt to determine whether or not a computer or computer program functioned well and to identify where the computer program might need improvement. However, such survey results are often unreliable because the surveys are frequently completed well after the activity was performed. In addition, survey participation rates can be low, and people may not provide accurate and honest answers to the survey.

In other cases of human-computer interaction, a person might not be using a software tool to accomplish a task, but instead might be consuming computer-accessed content or media such as news, pictures, music, or video. Currently, people consuming computer-driven content can tediously self-rate the media if they wish to communicate personal preferences. In some cases, viewers enter a specific number of stars corresponding to a level of like or dislike, while in other cases, users are asked to answer a list of questions. While a system for collecting users' evaluations of media and other products or services can be a helpful metric, current evaluation schemes are often tedious and challenging. Recommendations based on such a system of star rating and/or other means of self-reporting are imprecise, subjective, unreliable, and are further limited by sample size, as only a small number of viewers prove to actually take the time to rate the media they consume. Thus, in many cases, such subjective evaluation is neither a reliable nor practical way to evaluate personal responses to media.

A third-party observer can also be used to evaluate the human-computer interaction. A trained observer can often infer the user's mental state simply by observing the individual, their actions, and their context—e.g. their environment. The third party might also interact with the user and ask them questions about how they are feeling or details about what they are doing. While such a methodology can provide interesting results, the need for a trained observer to view and analyze the user means that using third-party observers is not scalable to large numbers of people performing many tasks in many different locations simultaneously. It also might be possible that the mere presence of the observer impacts the user's mental state, thus generating questionable results.

### SUMMARY

Mental state data, such as video of an individual's face, is captured on the individual and is useful for determining mental state information on that individual. The mental state data is intermittently collected. Additional data is also determined that is helpful in determining the mental state information, helps to interpret the mental state information, or otherwise provides information about mental states. The additional data is tagged to the mental state data and at least some of the mental state data is sent to a web service where it may be used to produce mental state information. A computer-implemented method for mental state analysis is disclosed comprising: receiving two or more portions of collected mental state data tagged with additional information, wherein the two or more portions of mental state data come from a plurality of sources of facial data, wherein the mental state data collected is intermittent in occurrence, and wherein the plurality of sources includes at least one computer-based device; interpolating the intermittent mental state data, wherein the interpolating is based on the additional information that was tagged; selecting one or more portions of the received two or more portions of mental state data based on the additional information that was tagged, wherein the one or more selected portions of mental state data are selected based, at least in part, on tags identifying a particular context; and analyzing, using one or more processors, the one or more selected portions of mental state data to generate mental state information. The one or more portions of mental state data are selected based, at least in part, on tags identifying a particular individual. The one or more portions of mental state data are selected based, at least in part, on tags identifying a particular context. The intermittent data that is interpolated is based, at least in part, on the additional information that was tagged. The result from the analyzing can be, at least in part, a mood measurement.

In embodiments, a computer-implemented method for mental state analysis comprises: capturing mental state data on an individual from a first source that includes facial information, wherein the mental state data collected is intermittent; capturing mental state data on the individual from at least a second source that includes facial data, wherein the at least a second source comprises a computer-based device; determining additional data about the mental state data wherein the additional data provides information about mental states and wherein the additional data includes information about a context as the mental state data was collected; tagging the additional data to the mental state data; interpolating the intermittent mental state data, wherein the interpolating is based on the additional data that was tagged; and sending at least a portion of the mental state data tagged with the additional data to a web service. The context may comprise an identity of another person in proximity of the individual.

In some embodiments, a computer-implemented method for mental state analysis comprises: contextual data being collected simultaneously with the mental state data. In embodiments, the contextual data includes location data, environmental information, or time data. In embodiments, the method further comprises evaluating a temporal signature for the mental states. In embodiments, the temporal signature is used to infer additional mental states. In embodiments, the method further comprises analyzing an emotional mood associated with the mental state information. In embodiments, analyzing the emotional mood is used to provide emotional health tracking. In embodiments, a computer program product embodied in a non-transitory computer readable medium for mental state analysis, the computer program product comprising code which causes one or more processors to perform operations of: capturing mental state data on an individual from a first source that includes facial information, wherein the mental state data collected is intermittent; capturing mental state data on the individual from at least a second source that includes facial data, wherein the at least a second source comprises a computer-based device; determining additional data about the mental state data wherein the additional data provides information about mental states and wherein the additional data includes information about a context as the mental state data was collected; tagging the additional data to the mental state data; interpolating the intermittent mental state data, wherein the interpolating is based on the additional data that was tagged; and sending at least a portion of the mental state data tagged with the additional data to a web service for processing.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram for mental state analysis.

FIG. 2 is a flow diagram for mental state and mood analysis.

FIG. 3 is a timeline with information tracks relating to mental states.

FIG. 4 shows mental state data with tags.

FIG. 5 is a flow diagram for mental state analysis.

FIG. 6 is a diagram for sensor analysis.

FIG. 7 illustrates feature extraction for multiple faces.

FIG. 8 shows live streaming of social video.

FIG. 9 shows example facial data collection including landmarks.

FIG. 10 shows example facial data collection including regions.

FIG. 11 is a flow diagram for detecting facial expressions.

FIG. 12 is a flow diagram for the large-scale clustering of facial events.

FIG. 13 shows unsupervised clustering of features and characterizations of cluster profiles.

FIG. 14A shows example tags embedded in a webpage.

FIG. 14B shows invoking tags to collect images.

FIG. 15 shows an example mood measurement display for individual activity.

FIG. 16 illustrates an example mood measurement dashboard display.

FIG. 17 illustrates example mood measurement statistical results.

FIG. 18 is a flow diagram for mental state-based recommendations.

FIG. 19 shows example image collection including multiple mobile devices.

FIG. 20 illustrates mental state analysis using connected computer-based devices.

FIG. 21 is a system diagram for mental state analysis.

### DETAILED DESCRIPTION

As a user interacts with a computing device, the user's mental state can provide valuable insight into the nature of the human-computer interaction. The mental state of the user can include such emotions as frustration, confusion, disappointment, hesitation, cognitive overload, fear, exhaustion, focus, engagement, attention, boredom, exploration, confidence, trust, delight, satisfaction, excitement, happiness, contentment, or many other human emotions. Instead of relying on imprecise self-reported user experience, understanding a user's mental state as he or she interacts with the computing device can prove valuable for a variety of reasons, including determining which aspects of a computer program are working well and which aspects need improvement; determining aspects of a computer game that are too difficult for some users or too easy for some users; measuring the effectiveness of advertisements; determining which parts of a video most please a specific user; or determining a user's preferences in order to better suggest what other media, games, or applications the specific user finds appealing, among other potential reasons.

While consuming media, the user can exhibit physical cues of his or her mental state, such as facial expressions, physiological reactions, and movements. Sensors coupled to a computer—depending on the embodiment, either the same computer with which the user is interacting or one or more other computers—can detect, capture, and/or measure one or more external manifestations of the user's mental state. In embodiments, for example, a still camera can capture images of the user's face; a video camera can capture images of the user's movements; a heart rate monitor can measure the user's heart rate; a skin conductance sensor can detect changes in the user's electrodermal activity response; and an accelerometer can measure such movements as gestures, foot tapping, or head tilts. Many other sensors and capabilities are possible. Some embodiments include multiple sensors to capture the user's mental state data.

Other data related to the mental state data can be determined; the identity of the individual being monitored, for example. Additionally, the task that the individual is performing or the media that the user is consuming can be identified, among other data points. A time, date, and/or location can be logged and surrounding environmental data such as temperature, humidity, lighting levels, noise levels, and the like, can be determined. Any number of other factors can be determined and tagged to the mental state data in order to associate the additional data with the mental state data. Tagging the additional data to the mental state data can be performed by including the additional data in the file that holds the mental state data. Any format can be used for the additional data, depending on file format used for the mental state data. Some examples of formats that can be used for the additional data include, but are not limited to, ID3, exchangeable image file format (EXIF), extensible metadata platform (XMP), or other metadata standards. By tagging the mental state data with the additional data, the additional data is persistently associated with the mental state data.

Once the mental state data has been collected and tagged with the additional data, at least some of the tagged mental state data is sent to a web service. The web service can comprise a computer that is communicated with over a network or through a network of networks, such as the internet. The web service receives the tagged mental state data and selects some of the tagged mental state data based on the additional data included in the tags. The web service then analyzes the selected tagged mental state data to infer mental state information about the individual. The mental state information is then used to analyze or render an output, either by the web service or by another computer that receives the mental state information from the web service. In some embodiments, the rendering is performed on the computer hosting the web service, while in other embodiments, the rendering is either executed on the computer that originally collected the mental state data or on a different computer. In some embodiments, the output rendered as a result of the analysis is a mood measurement of the individual. The mood measurement may then be further rendered to produce output for use by the individual, by another computer ore web service, or by the computer-based device on which the mental state data or some of the mental state data was captured. The second or more information sources which comprises a computer-based device includes computing power as evidenced typically by one or more processors able to execute code and responsive to sophisticated, interactive user control. A mobile phone, typically of the smartphone variety, would be a computer-based device. Likewise, a tablet or a laptop computer would be a computer-based device. A passive monitor, such as a baby monitor or an eldercare monitor, would not typically be a computer-based device, even if the monitor provided basic features such as sound, video, channel selection, on/off switch, volume control, etc.

The rendered output can include text, icons, pictures, graphs, binary data, or any other form or output that, depending on the embodiment, can be interpreted by a person or another computer. In at least one embodiment, the rendered output includes a graph showing the prevalence over time of a particular mental state. In some embodiments, the rendered output includes an icon that changes based on the user's mental state. In some embodiments, the rendered output includes a file containing numerical data based on the obtained analysis. The result of the mental state analysis can also be included in a calendar where it can be displayed or compared with the ongoing activities already included in the calendar.

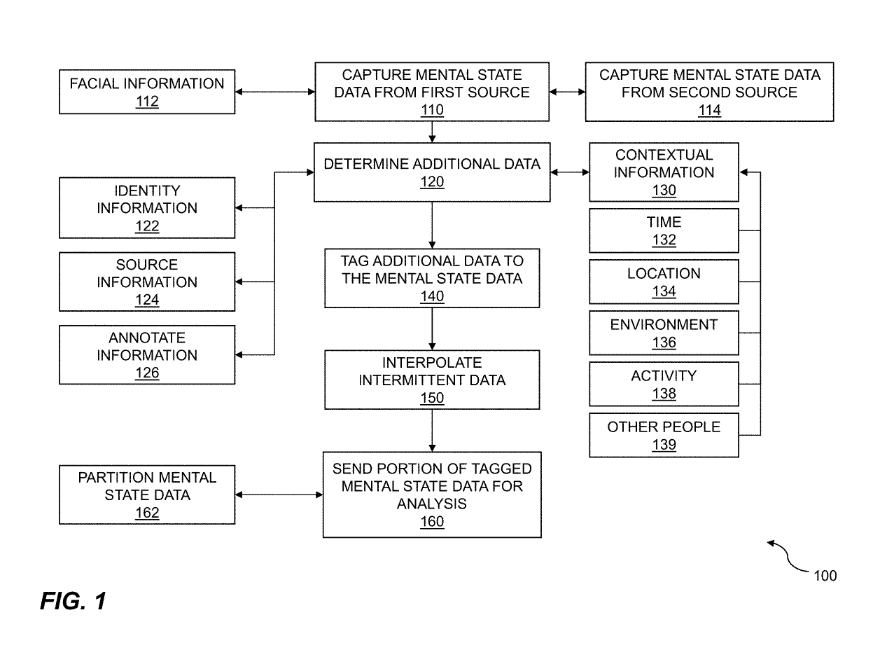

FIG. 1 is a flow diagram 100 for mental state analysis. The flow 100 describes a computer-implemented method for mental state analysis that includes capturing mental state data on an individual from a first source 110. The mental state data includes still images or video of the individual in some embodiments; these images or video can include the individual's face. Thus, the mental state data from the first source can include facial information 112. In some embodiments, the mental state data includes data from biosensors or other types of mental state data. The flow 100 comprises, in some embodiments, obtaining mental state data from a second source 114 where the mental state data from the second source can include facial information. In embodiments, mental state data is collected from multiple devices while a user is performing a computer-based task using an electronic display during a portion of time. In embodiments, the multiple devices include a tablet computer or a cell phone.

The flow 100 also includes determining additional data 120 about the mental state data. The additional data can be information about the identity 122 of the individual, information about the source 124 of the mental state data, contextual information 130, or other metadata. In other words, the additional data can include information on a source 124 that collected the mental state data, and/or the context 130 in which the mental state data was collected. In some embodiments, the context comprises a time 132, while in other embodiments the context comprises a location 134. The location can be determined by any mechanism including, but not limited to, internet protocol (IP) address mapping, manual entering of location data by an operator or the individual being monitored, or by receiving radio broadcasts. In at least one embodiment, the location is determined using GPS. The location can be identified using any type of identification including, but not limited to, an address, latitude and longitude coordinates, a building identifier, or a room identifier. Practically, the location information could identify a building, a room, or another type of address or position. Additionally, in some embodiments the context comprises environmental information 136.

In many embodiments, the context comprises an activity 138 performed by the individual. The activity includes at least one of talking on a phone, playing a videogame, working at a computer, or watching a media presentation, depending on the embodiment. The context can further include information further identifying the context, such as the name or number of the other party on the phone, the name of the videogame, a descriptor of the activity being performed within the videogame, the type of activity being worked on with the computer, the name of the media presentation being watched, or other descriptors. In other embodiments, the context comprises an identity for another person or other people 139 within a given proximity of the individual. The additional data can include human coded information on the individual. The additional data can be annotated information 126 using the human coded information. The human coded information can include analysis of mental states seen by the human coder in the face of the individual. The human coded information can include a summary of the mental state analysis.

The additional data can include information about an identity of the individual 122. The information about the individual can be in any form, but in some embodiments the information about the identity of the individual includes a name of the individual and/or an identity value for the individual. The identity of the individual can be determined by any method, including, but not limited to, manual entry by the individual or an operator, a computer system account login, or an identity card scan. In at least one embodiment, the information about the identity of the individual is determined using face recognition. In at least one embodiment, the portion of the mental state data is determined based on face recognition.

The various data and additional data from multiple sources can be synchronized. In some embodiments, the data and additional data can include timestamps for synchronizing. In other cases, a repetitive pulse can be used to align information as needed which can be, in some cases, an audio or light pulse or group of such pulses. These pulses can be used for later alignment of the data and additional data during analysis.

The flow 100 includes tagging the additional data to the mental state data 140. Through such tagging, additional data can be associated with the mental state data. The tagging can be done by any method for associating data in a computer system. By tagging the mental state data with the additional data, the additional data is associated with the mental state data in a persistent manner. In some embodiments, the additional data can be included in the file that holds the mental state data. Depending on file format used for the mental state data, any format can be used for the additional data. Some examples of formats that can be used for the additional data include ID3, exchangeable image file format (EXIF), extensible metadata platform (XMP), or any other metadata standard. In other embodiments, the additional data is stored in a separate file linked to the file that holds the mental state data. In yet other embodiments, a separate file contains links to both the additional data file and the mental state data file.

The flow 100 includes interpolating intermittent mental state data 150. In embodiments, interpolating mental state data when the collected mental state data is intermittent, and/or imputing additional mental state data where the mental state data is missing. The mental state data may be intermittent or missing when there is any kind of gap in the data source providing the mental state data. For example, if the source of the mental state data is the front-facing camera of a cell phone on which an individual is watching a video, the facial data from the camera can be intermittent or missing any time the individual looks away, is distracted, covers the camera, experiences changing lighting conditions, steps onto a bus or train, gets jostled, and any number of intermittent or missing data scenarios. In some embodiments, greater than 25% of the potential mental state data is missing or intermittent. In many embodiments, between 5% and 25% of the data is missing or intermittent. Interpolating the intermittent data and/or imputing the missing data is a key component in providing complete mental state data for an accurate analysis.

The flow 100 includes sending at least a portion of the mental state data tagged with the additional data to a web service 160. The web service can be contacted over a network or a network of networks—such as the Internet—and the web service can be hosted on a different computer than the computer used to capture the mental state data. The mental state data can be partitioned 162 based on the additional data. The portion of the mental state data to be sent to the web service can be determined based on the additional data. In one embodiment, the tagged mental state data is examined and the portions which are tagged with the identity of a particular individual are sent. In other embodiments, the portions that are tagged with a particular activity are sent. Other embodiments utilize different tags to determine which of the portions of the mental state date are to be sent. In at least one embodiment, the portion of the mental state data to be sent to the web service is determined based on facial recognition performed on the mental state data when the mental state data is comprised of facial images. The mental state data can be combined with data from other sources, such as social media information, to augment the mental state analysis. In some cases a user can obtain feedback based on the mental state data in order to enhance an experience for the user.

The flow 100 can further comprise analyzing the mental state data to produce mental state information. In some embodiments, the analyzing is performed based on facial movement. Other embodiments analyze biosensor data to produce mental state information. Various mental states can be inferred, such as frustration, confusion, disappointment, hesitation, cognitive overload, fear, exhaustion, focus, engagement, attention, boredom, exploration, confidence, trust, delight, satisfaction, excitement, happiness, sadness, stress, anger, contentment, or many other human emotions. In some embodiments, the additional data is used in conjunction with the mental state data to produce the mental state information. In some embodiments, the additional data is used to limit the mental state data that is analyzed. In other embodiments, the additional data directly contributes to the determining of the mental state, such as by analyzing the contents of an email being read. Various steps in the flow 100 may be changed in order, repeated, omitted or the like without departing from the disclosed concepts. Various embodiments of the flow 100 may be included in a computer program product embodied in a non-transitory computer readable medium that includes code executable by one or more processors.

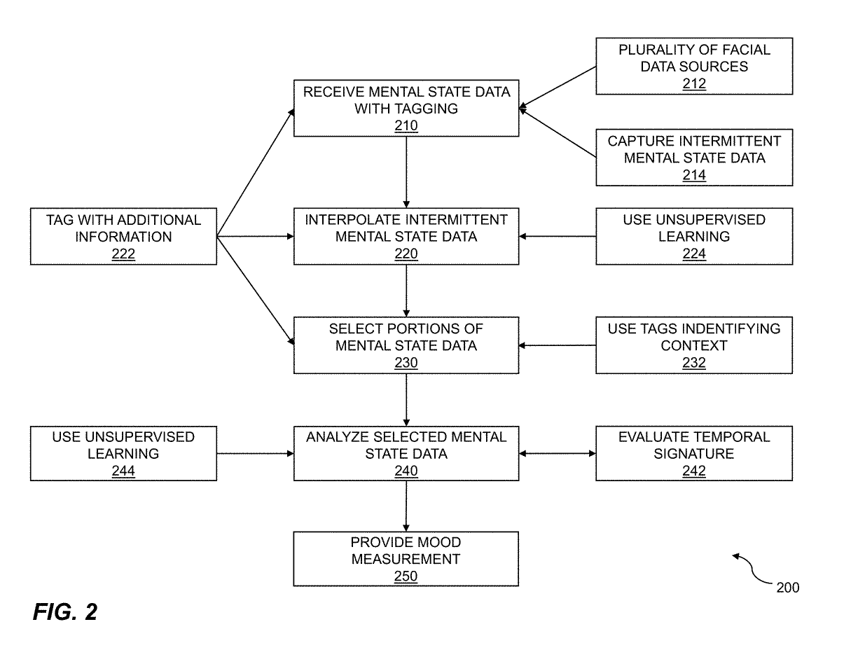

FIG. 2 is a flow diagram for mental state and mood analysis. The flow 200 includes receiving mental state data with tagging 210. The received data can be two or more portions of collected mental state data tagged with additional information 222. The additional information can identify a particular individual, can identify one or more contexts, can identify at least two different timestamps, and so on. The contextual data can include location data, environmental information, time data, and so on. The contextual data can be collected simultaneously with the mental state data. The received data can be from captured mental state data that is intermittent 214. The received data can be from a plurality of sources of facial data 212. The plurality of sources can include data on the individual and/or on multiple individuals. The flow 200 includes interpolating intermittent mental state data 220. The interpolating may actually be occurring in times when the mental state data is so intermittent that it appears to be missing. For example, after a particularly successful segment of a video game, the individual may celebrate for a number of seconds, say, from a half second to ten seconds, and thus the facial data may be completely missing for that time. The interpolating may be a straight-line interpolation between mental state data inputs, or a more sophisticated means, such as unsupervised learning 224, may be used. Unsupervised learning can take several occurrences of intermittent mental state data and provide interpolation based on the tagging with additional information 222. Thus it is apparent that a simple interpolation will provide the same result every time, whereas interpolation based on unsupervised learning may provide different results over time, with the differences providing more accurate interpolations based on the mental state data received along with the additional contextual data that is tagged to the mental state data. In embodiments, performing unsupervised learning can enable the interpolating based on the additional data that was tagged.

The flow 200 includes selecting portions of mental state data 230. The selected portions can be based on the additional information that is tagged 222. In some embodiments, the additional information used for selection is, at least in part, tags identifying context 232. For example, the context of a time stamp can be used to select appropriate portions of the two or more portions for further analysis. The flow 200 includes analyzing the selected mental state data 240. The analysis of the one or more selected portions of mental state data can be used to generate mental state information, which is organized and interpreted mental state data. For example, the mental state data of rolled eyes followed by a frown, may, in the context of a difficult game level, generate a mental state of frustration.

The analysis of mental state data can use unsupervised learning 244. The unsupervised learning can include learning additional data about the mental state data, wherein the learning is based on mental state information and mental state information collection context. The analysis can involve evaluating a temporal signature 242. A temporal signature is a representation of one or more elements, such as mental state data, according to their occurrence in the time domain. For example, a happy scene in a movie that suddenly turns scary would produce a temporal signature of the mental state data that represents the emotional content of an individual's response to a sudden stimulus. Detecting certain temporal signatures in certain sequences can then be used to analyze selected mental state data. The flow 200 can include providing a mood measurement 250 as a result from the analysis. The mood measurement can reflect a single mental state or reflect a composite of several mental states, along with the temporal timing, length, and strength of the several mental states. For example, an inferred mental state of attentiveness may result in outputting a mood of being satisfied, whereas an inferred mental state of attentiveness along with increased arousal and positive valence may result in outputting a mood of being happy. In embodiments, the method further comprises analyzing an emotional mood associated with the mental state information. In embodiments, analyzing the emotional mood is used to provide emotional health tracking. Various steps in the flow 200 may be changed in order, repeated, omitted or the like without departing from the disclosed concepts. Various embodiments of the flow 200 may be included in a computer program product embodied in a non-transitory computer readable medium that includes code executable by one or more processors.

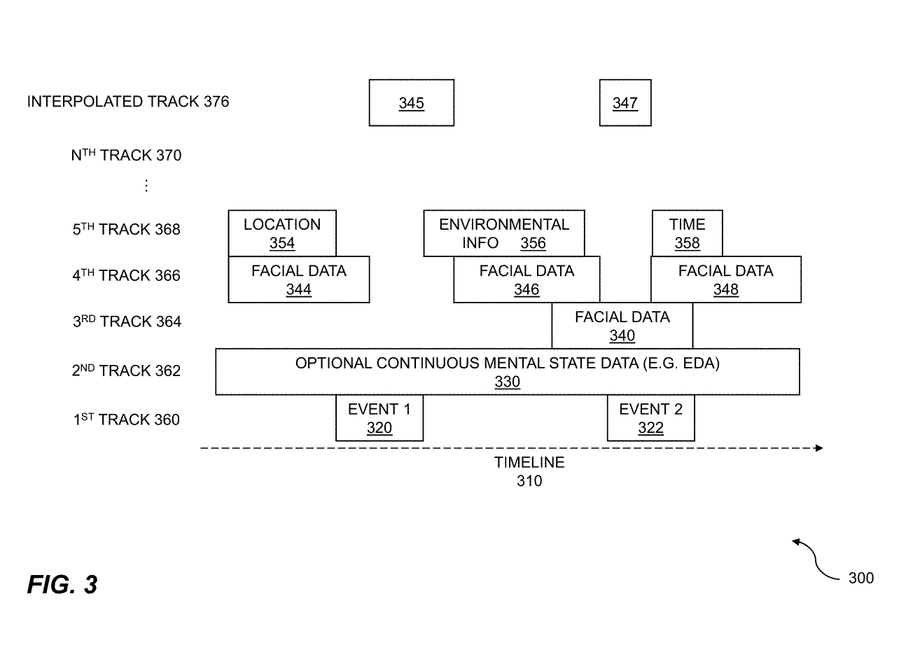

FIG. 3 is a timeline 310 with information tracks 300 relating to mental states. A first track 360 shows events which can be related to the individual's use of a computer. A first event 320 marker can indicate an action that the individual took (such as launching an application); an action initiated by the computer (such as the presentation of a dialog box); an external event (such as a new global positioning system [GPS] coordinate); or an event such as the receipt of an e-mail, a phone call, a text message, or the like. In some embodiments, a photograph can be used to document an event or simply save contextual information in the first track 360. A second event 322 marker can indicate another action or event. Such event markers can be used to provide contextual information and can also include such things as copies of emails, text messages, phone logs, file names, or other information that can prove useful in understanding the context of a user's actions.

A second track 362 can include continuously collected mental state data such as electrodermal activity data 330. A third track 364 can include facial data 340, which can be collected on an intermittent or continuous basis by a first camera, such as a room camera or a front-side camera of a mobile, or cell, phone. Thus, the mental state data from the first source can include facial information. The facial data can be collected intermittently, for example, only when the individual is facing a camera. The facial data 340 can include one or more still photographs, videos, or abstracted facial expressions which can be collected when the user looks in the direction of the camera. A fourth track 366 can include facial data collected on an intermittent or continuous basis by a second camera, such as a tablet webcam used for watching a video or playing a computer game. The fourth track can include three instances of collected facial data 344, 346, and 348. The three collected instances of facial data 344, 346, and 348 can include one or more still photographs, videos, or abstracted facial expressions which can be collected when the user looks in the direction of a camera.

A fifth track 368 can include contextual data collected simultaneously with the mental state data. In one example, the fifth track 368 includes location data 354, environmental information 356, and time data 358, although other types of contextual data can be collected in other embodiments. In the embodiment shown, the fifth track 368 of contextual data can be associated with the fourth track 366 of mental state data. Some embodiments determine multiple tracks of additional data that can be associated with one or more tracks of mental state data. For example, another track can include identity information of the individual being monitored by the camera capturing the third track 364 of mental state data.

Additional tracks—in the timeline shown, through the nth track 370—of mental state data or additional data of any type can be collected. The additional tracks can be collected on a continuous or on an intermittent basis. The intermittent basis can be either occasional or periodic. Analysis can further comprise interpolating mental state data when the collected mental state data is intermittent, and/or imputing additional mental state data where the mental state data is missing. One or more interpolated tracks 376 can be included and can be associated with mental state data that is collected on an intermittent basis, such as the facial data of the fourth track 366. The two instances of interpolated data, interpolated data 345 and 347, can contain interpolations of the facial data of the fourth track 366 for the time periods where no facial data was collected in that track. Other embodiments interpolate and/or impute data for periods where no track includes facial data. In other embodiments, analysis includes interpolating mental state analysis when the collected mental state data is intermittent.

The mental state data, such as the continuous mental state data 330 and/or any of the collected facial data can be tagged. In the example timeline shown, facial data 340, 344, 346, and 348 are tagged. The tags can include metadata related to the mental state data, including, but not limited to, the device that collected the mental state data; the individual from whom the mental state data was collected; the task being performed by the individual; the media being viewed by the individual; and the location, the environmental conditions, the time, the date, or any other contextual information useful for mental state analysis. The tags can be used to locate pertinent mental state data; for example, the tags can be used to identify useful mental state data for retrieval from a database. The tags can be included with the mental state data that is sent over the internet to cloud or web-based storage and/or services and can be used remotely, but the tags can also be used locally on the machine where the mental state data was collected.

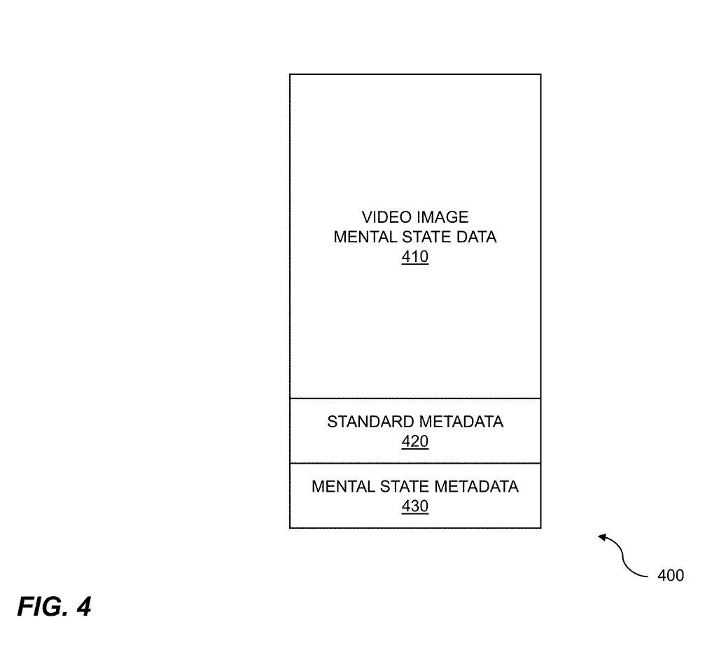

FIG. 4 shows mental state data with tags 400. The mental state data with tags 400 includes video image mental state data 410 captured on an individual from a first source. In some embodiments, the source of the mental state data includes certain standard metadata 420 with the mental state data 410. For example, a video camera which includes timestamps along with video data demonstrates such metadata inclusion. A still camera which includes EXIF data identifying the camera model, exposure information, and day and date information in the JPEG or other image file format containing the compressed image data shows another instance of metadata inclusion.

In embodiments, additional data which provides information about the mental state data 410 is determined. Such additional data can be tagged to the mental state data as mental state metadata 430. The mental state metadata 430 can provide information about the mental states useful in the analysis of the mental state data 410. The mental state metadata 430, or additional data, is data that is not tagged to the mental state data by the source of the mental state data and not always known to the source of the mental state data 410. Thus, the mental state metadata 430 is tagged to the mental state data 410 by an entity that is not the original source of the mental state data.

In one embodiment, a video camera is used to capture the mental state data 410. The video camera can include standard metadata 420 such as time and date and model number of the camera, along with the video image, which in this case comprises video image mental state data 410, in a MPEG-4 data stream that is sent from the video camera to a mental state data collection machine. The standard metadata 420 can be included using standard metadata formats defined by the MPEG-4 specification. The mental state data collection machine can determine an identity of the individual being monitored, such as a login ID, and an activity of that individual, such as watching a particular media presentation. The mental state data collection machine can then tag the video image with the login ID and the name of the particular media presentation as mental state metadata 430. In at least one embodiment, the mental state data collection machine formats the mental state metadata as XMP metadata and includes it in the MPEG-4 file. Other embodiments determine different additional information to be used as mental state metadata 430 and use different formats to tag the mental state data 410 with the mental state metadata 430.

Once the data collection machine has captured mental state data, at least a portion of the mental state data tagged with the additional data is sent to a web service. The portion of the mental state data sent to the web service can be based on the additional contextual data collected, or can be based on mental state metadata 430. At the web service, portions of mental state data can be selected for analysis based, at least in part, on tags identifying one or more contexts. In at least one embodiment, the selected portions are based, at least in part, on identifying a particular individual. In some embodiments, the selected portions include tags identifying at least two different timestamps so that samples can be distributed over a period of time. In at some embodiments, the selected portions are based, at least in part, on tags identifying a particular context. Once the portions are selected, they can be analyzed by the web service and used to create mental state information.

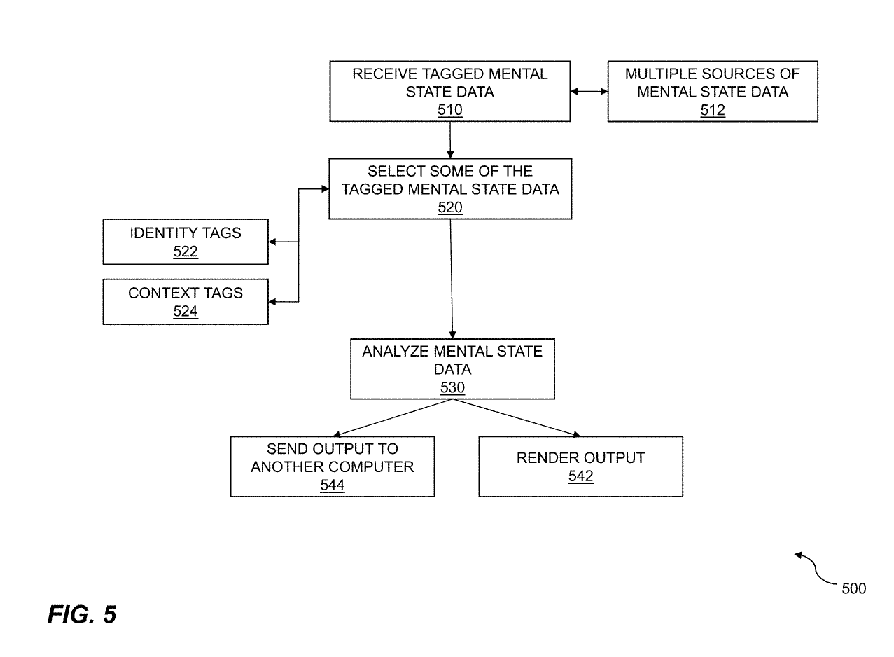

FIG. 5 is a flow diagram 500 for mental state analysis. The flow 500 describes a computer-implemented method for mental state analysis where tagged mental state data is received. The flow 500 includes receiving two or more portions of mental state data tagged with additional information 510. In some embodiments, the mental state data is received from multiple sources 512, so the two or more portions can come from a plurality of sources of facial data. In some cases, analysis can be performed to evaluate mental state data measurements across various devices from which the mental state data is received. The flow 500 continues by selecting one or more portions of the received two or more portions of mental state data 520, based on the additional data from the tags. In some embodiments, the one or more portions of mental state data are selected based, at least in part, on tags identifying a particular individual 522, or the one or more portions of mental state data can be selected based, at least in part, on tags identifying one or more contexts 524. Context information can be used to narrow the focus to a particular area of analysis. Likewise, context information can be used to exclude certain events from mental state analysis. For instance, any mental state data associated with unintended noise or distraction can be tagged to be ignored during later analysis. In some embodiments, the one or more portions of mental state data are selected to include tags identifying at least two different timestamps. The one or more portions of mental state data can be selected based, at least in part, on tags identifying a particular context comprising a particular location, a particular activity, or the like. Any algorithm can be used to select the one or more portions for analysis.

The flow 500 continues by analyzing the one or more selected portions of mental state data 530 to generate mental state information wherein a result from the analyzing is used to render an output on mental states 542. Analysis and rendering, based on tagged data, can aid a human user in being able to focus on areas of particular interest without wading through enormous sums of irrelevant data. A rendering can include a summary of mental states, a graphical display showing a media presentation and associated mental states, a social media pages with mental state information, and the like. The rendering can also include excerpts of a media presentation such as, in some embodiments, a highlight-reel style presentation based on mental states and associated tagged data. In some cases portions of a media presentation can be excluded based on mental states and tagged data. In some embodiments, the same computer that was used to analyze the mental state data 530 is also used to render the output, but in other embodiments, the output used for rendering being sent to another computer 544 where the other computer provides the rendering. The rendering can be any type of rendering including textual rendering, graphical rendering, pictorial rendering, or a combination thereof. In some embodiments, another computer can provide information to another user. This other user can perform various analyses including AB type testing and comparisons. Various steps in the flow 500 may be changed in order, repeated, omitted, or the like without departing from the disclosed concepts. Various embodiments of the flow 500 may be included in a computer program product embodied in a non-transitory computer readable medium that includes code executable by one or more processors.

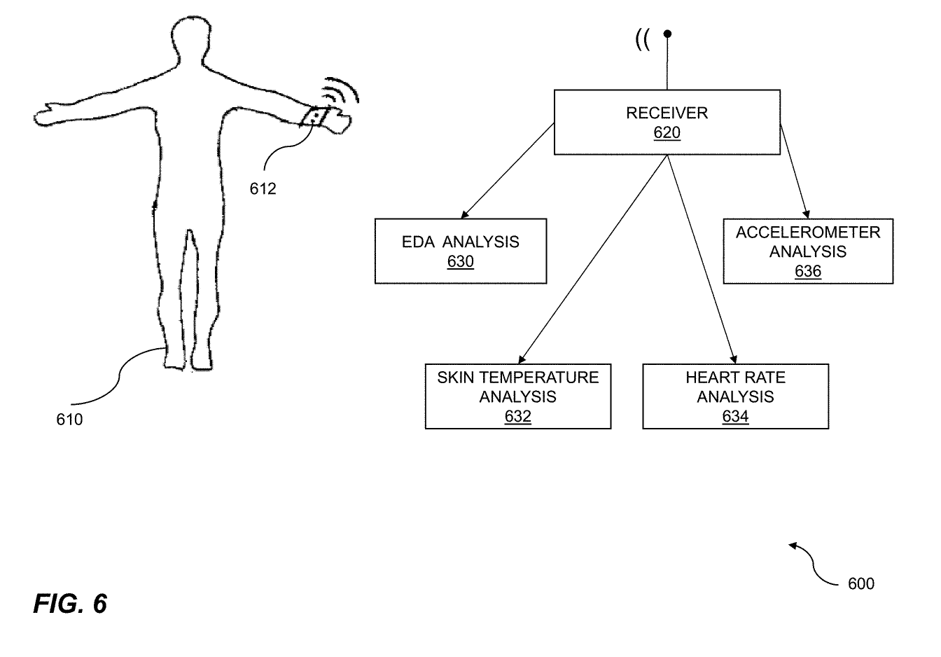

FIG. 6 is a diagram for sensor analysis. A system 600 can analyze data collected from a person 610 as he or she interacts with a computer or views a media presentation. The person 610 can have a biosensor 612 attached to him or her for the purpose of collecting mental state data and biosensor information. The biosensor 612 can be placed on the wrist, palm, hand, head, or another part of the body. In some embodiments, multiple biosensors are placed on the body in multiple locations. The biosensor 612 can include detectors for physiological data such as electrodermal activity, skin temperature, accelerometer readings, and the like. Other detectors for physiological data can also be included, such as heart rate, blood pressure, EKG, EEG, other types of brain waves, and other physiological detectors. The biosensor 612 can transmit collected information to a receiver 620 using wireless technology such as Wi-Fi, Bluetooth®, 802.11, cellular, or other bands. In other embodiments, the biosensor 612 communicates with the receiver 620 using other methods such as a wired or optical interface. The receiver can provide the collected data to one or more components in the system 600. In some embodiments, the biosensor 612 records multiple types of physiological information in memory for later download and analysis. In some embodiments, the download of recorded physiological data is accomplished through a USB port or another form of wired or wireless connection.

Mental states can be inferred based on physiological data such as the physiological data collected from the sensor 612. Mental states can also be inferred based on facial expressions and head gestures observed by a webcam, or by using a combination of data from the webcam and data from the sensor 612. The mental states can be analyzed based on arousal and valence. Arousal can range from being highly activated, such as when someone is agitated, to being entirely passive, such as when someone is bored. Valence can range from being very positive, such as when someone is happy, to being very negative, such as when someone is angry. Physiological data can include one or more of electrodermal activity (EDA), heart rate, heart rate variability, skin temperature, respiration, accelerometer readings, and other types of analysis of a human being. It should be understood that both here and elsewhere in this document, physiological information can be obtained either by biosensor 612 or by facial observation via an image capturing device. Facial data can include facial actions and head gestures used to infer mental states. Further, the data can include information on hand gestures, body language, and body movements such as visible fidgets. In some embodiments, these movements are captured by cameras, while in other embodiments, these movements are captured by sensors. Facial data can include tilting of the head to the side, leaning forward, smiling, frowning, and many other gestures or expressions.

In some embodiments, electrodermal activity is collected, either continuously, every second, multiple times per second, or on some other periodic basis. Alternatively, electrodermal activity can be collected on an intermittent basis. The electrodermal activity can be recorded and stored onto a disk, a tape, flash memory, or a computer system, or can be streamed to a server. The electrodermal activity can be analyzed 630 to indicate arousal, excitement, boredom, or other mental states based on observed changes in skin conductance. Skin temperature can be collected and/or recorded on a periodic basis. In turn, the skin temperature can be analyzed 632. Changes in skin temperature can indicate arousal, excitement, boredom, or other mental states. Heart rate information can also be collected, recorded, and analyzed 634. A high heart rate can indicate excitement, arousal, or other mental states. Accelerometer data can be collected and used to track one, two, or three dimensions of motion. The accelerometer data can be recorded. The accelerometer data can be used to create an actigraph showing an individual's activity level over time. The accelerometer data can be analyzed 636 and can indicate a sleep pattern, a state of high activity, a state of lethargy, or other states. The various data collected by the biosensor 612 can be used along with the facial data captured by the webcam in the analysis of mental state. Contextual information can be based on one or more of skin temperature and accelerometer data. The mental state data can include one or more of a group including physiological data, facial data, and accelerometer data.

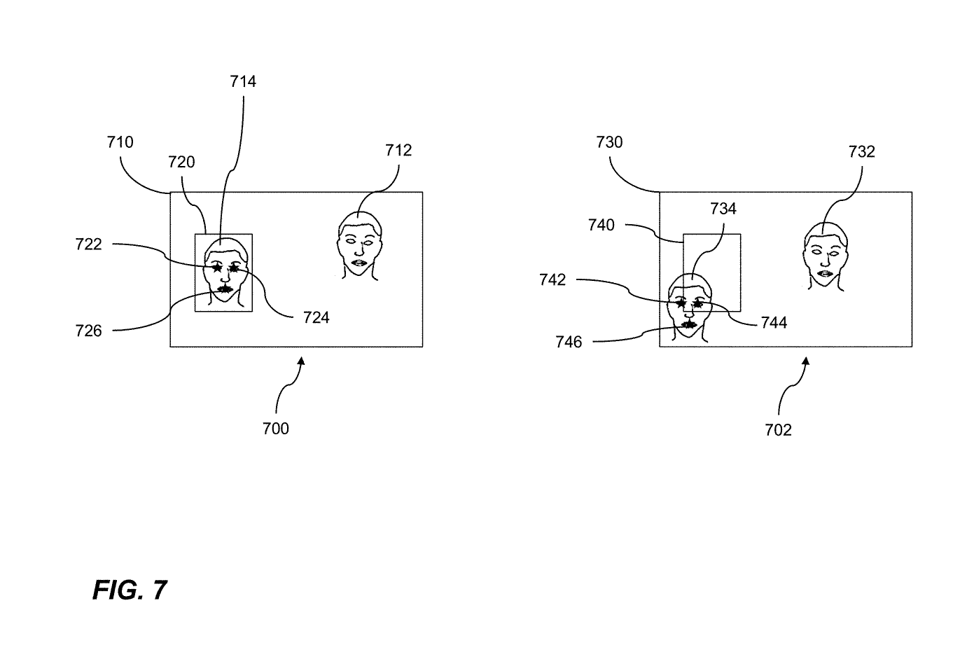

FIG. 7 illustrates feature extraction for multiple faces. The features can be evaluated within tagging and mood analysis of mental state data collected from multiple sources. The feature extraction for multiple faces can be performed for faces that can be detected in multiple images. The images can be analyzed for mental states and/or facial expressions. A plurality of images can be received of an individual viewing an electronic display. A face can be identified in an image, based on the use of classifiers. The plurality of images can be evaluated to determine mental states and/or facial expressions of the individual. The feature extraction can be performed by analysis using one or more processors, using one or more video collection devices, and by using a server. The analysis device can be used to perform face detection for a second face, as well as for facial tracking of the first face. One or more videos can be captured, where the videos contain one or more faces. The video or videos that contain the one or more faces can be partitioned into a plurality of frames, and the frames can be analyzed for the detection of the one or more faces. The analysis of the one or more video frames can be based on one or more classifiers. A classifier can be an algorithm, heuristic, function, or piece of code that can be used to identify into which of a set of categories a new or particular observation, sample, datum, etc. should be placed. The decision to place an observation into a category can be based on training the algorithm or piece of code, by analyzing a known set of data, known as a training set. The training set can include data for which category memberships of the data can be known. The training set can be used as part of a supervised training technique. If a training set is not available, then a clustering technique can be used to group observations into categories. The latter approach, or unsupervised learning, can be based on a measure (i.e. distance) of one or more inherent similarities among the data that is being categorized. When the new observation is received, then the classifier can be used to categorize the new observation. Classifiers can be used for many analysis applications including analysis of one or more faces. The use of classifiers can be the basis of analyzing the one or more faces for gender, ethnicity, and age; for detection of one or more faces in one or more videos; for detection of facial features, for detection of facial landmarks, and so on. The observations can be analyzed based on one or more of a set of quantifiable properties. The properties can be described as features and explanatory variables and can include various data types that can include numerical (integer-valued, real-valued), ordinal, categorical, and so on. Some classifiers can be based on a comparison between an observation and prior observations, as well as based on functions such as a similarity function, a distance function, and so on.

Classification can be based on various types of algorithms, heuristics, codes, procedures, statistics, and so on. Many techniques exist for performing classifications. This classification of one or more observations into one or more groups can be based on distributions of the data values, probabilities, and so on. Classifiers can be binary, multiclass, linear, and so on. Algorithms for classification can be implemented using a variety of techniques, including neural networks, kernel estimation, support vector machines, use of quadratic surfaces, and so on. Classification can be used in many application areas such as computer vision, speech and handwriting recognition, and so on. Classification can be used for biometric identification of one or more people in one or more frames of one or more videos.

Returning to FIG. 7, the detection of the first face, the second face, and multiple faces can include identifying facial landmarks, generating a bounding box, and prediction of a bounding box and landmarks for a next frame, where the next frame can be one of a plurality of frames of a video containing faces. A first video frame 700 includes a frame boundary 710, a first face 712, and a second face 714. The video frame 700 also includes a bounding box 720. Facial landmarks can be generated for the first face 712. Face detection can be performed to initialize a second set of locations for a second set of facial landmarks for a second face within the video. Facial landmarks in the video frame 700 can include the facial landmarks 722, 724, and 726. The facial landmarks can include corners of a mouth, corners of eyes, eyebrow corners, the tip of the nose, nostrils, chin, the tips of ears, and so on. The performing of face detection on the second face can include performing facial landmark detection with the first frame from the video for the second face and can include estimating a second rough bounding box for the second face based on the facial landmark detection. The estimating of a second rough bounding box can include the bounding box 720. Bounding boxes can also be estimated for one or more other faces within the boundary 710. The bounding box can be refined, as can one or more facial landmarks. The refining of the second set of locations for the second set of facial landmarks can be based on localized information around the second set of facial landmarks. The bounding box 720 and the facial landmarks 722, 724, and 726 can be used to estimate future locations for the second set of locations for the second set of facial landmarks in an upcoming video frame from the first video frame.

A second video frame 702 is also shown. The second video frame 702 includes a frame boundary 730, a first face 732, and a second face 734. The second video frame 702 also includes a bounding box 740 and the facial landmarks 742, 744, and 746. In other embodiments, multiple facial landmarks are generated and used for facial tracking of the two or more faces of a video frame, such as the shown second video frame 702. Facial points from the first face can be distinguished from other facial points. In embodiments, the other facial points include facial points of one or more other faces. The facial points can correspond to the facial points of the second face. The distinguishing of the facial points of the first face and the facial points of the second face can be used to distinguish between the first face and the second face, to track either the first face or second face, both the first and the second faces, and so on. Other facial points can correspond to the second face. As mentioned above, multiple facial points can be determined within a frame. One or more of the other facial points that are determined can correspond to a third face. The location of the bounding box 740 can be estimated, where the estimating can be based on the location of the generated bounding box 720 shown in the first video frame 700. The three facial landmarks shown, facial landmarks 742, 744, and 746, might lie within the bounding box 740 or might not lie partially or completely within the bounding box 740. For instance, the second face 734 might have moved between the first video frame 700 and the second video frame 702. Based on the accuracy of the estimating of the bounding box 740, a new estimation can be determined for a third, future frame from the video, and so on. The evaluation can be performed, all or in part, on semiconductor-based logic.

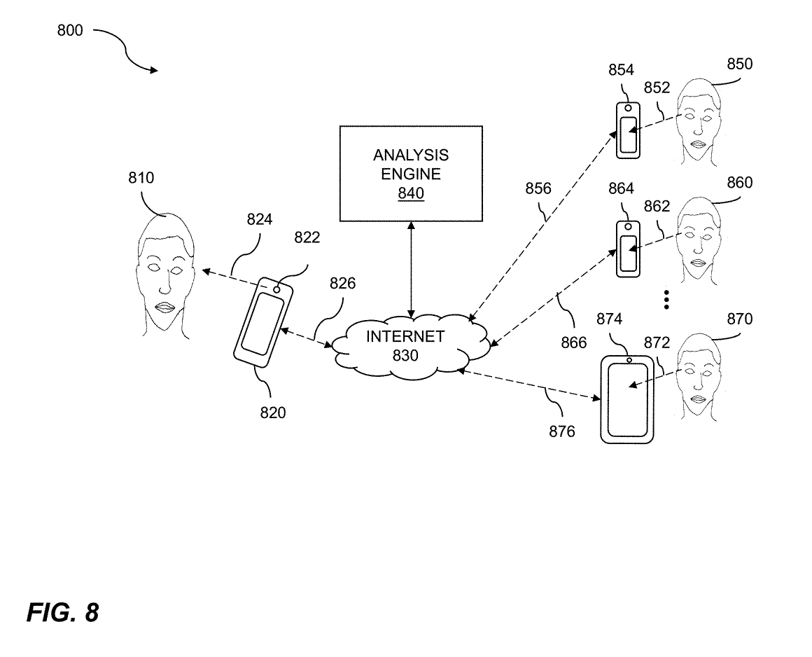

FIG. 8 shows live streaming of social video. The living streaming can be used within tagging and mood analysis of mental state data collected from multiple sources. Analysis of live streaming of social video can be performed using data collected from evaluating images to determine a facial expression and/or mental state. A plurality of images of an individual viewing an electronic display can be received. A face can be identified in an image, based on the use of classifiers. The plurality of images can be evaluated to determine facial expressions and/or mental states of the individual. The streaming and analysis can be facilitated by a video capture device, a local server, a remote server, a semiconductor-based logic, and so on. The streaming can be live streaming and can include mental state analysis, mental state event signature analysis, etc. Live streaming video is an example of one-to-many social media, where video can be sent over the Internet from one person to a plurality of people using a social media app and/or platform. Live streaming is one of numerous popular techniques used by people who want to disseminate ideas, send information, provide entertainment, share experiences, and so on. Some of the live streams can be scheduled, such as webcasts, online classes, sporting events, news, computer gaming, or video conferences, while others can be impromptu streams that are broadcasted as needed or when desirable. Examples of impromptu live stream videos can range from individuals simply wanting to share experiences with their social media followers, to live coverage of breaking news, emergencies, or natural disasters. The latter coverage is known as mobile journalism and is becoming increasingly common. With this type of coverage, “reporters” can use networked, portable electronic devices to provide mobile journalism content to a plurality of social media followers. Such reporters can be quickly and inexpensively deployed as the need or desire arises.

Several live streaming social media apps and platforms can be used for transmitting video. One such video social media app is Periscope™ that can transmit a live recording from one user to that user's Periscope™ account and other followers. The Periscope™ app can be executed on a mobile device. The user's Periscope™ followers can receive an alert whenever that user begins a video transmission. Another live-stream video platform is Twitch™ that can be used for video streaming of video gaming and broadcasts of various competitions and events.

The example 800 shows a user 810 broadcasting a video live-stream to one or more people as shown by the person 850, the person 860, and the person 870. A portable, network-enabled electronic device 820 can be coupled to a front-facing camera 822. The portable electronic device 820 can be a smartphone, a PDA, a tablet, a laptop computer, and so on. The camera 822 coupled to the device 820 can have a line-of-sight view 824 to the user 810 and can capture video of the user 810. The captured video can be sent to a recommendation or analysis engine 840 using a network link 826 to the Internet 830. The network link can be a wireless link, a wired link, and so on. The analysis engine 840 can recommend to the user 810 an app and/or platform that can be supported by the server and can be used to provide a video live stream to one or more followers of the user 810. In the example 800, the user 810 has three followers: the person 850, the person 860, and the person 870. Each follower has a line-of-sight view to a video screen on a portable, networked electronic device. In other embodiments, one or more followers follow the user 810 using any other networked electronic device, including a computer. In the example 800, the person 850 has a line-of-sight view 852 to the video screen of a device 854; the person 860 has a line-of-sight view 862 to the video screen of a device 864, and the person 870 has a line-of-sight view 872 to the video screen of a device 874. The portable electronic devices 854, 864, and 874 can each be a smartphone, a PDA, a tablet, and so on. Each portable device can receive the video stream being broadcasted by the user 810 through the Internet 830 using the app and/or platform that can be recommended by the analysis engine 840. The device 854 can receive a video stream using the network link 856, the device 864 can receive a video stream using the network link 866, the device 874 can receive a video stream using the network link 876, and so on. The network link can be a wireless link, a wired link, a hybrid link, and so on. Depending on the app and/or platform that can be recommended by the analysis engine 840, one or more followers, such as the followers 850, 860, 870, and so on, can reply to, comment on, and otherwise provide feedback to the user 810 using their devices 854, 864, and 874, respectively. In embodiments, mental state and/or facial expression analysis is performed on each follower (850, 860, and 870). An aggregate viewership score of the content generated by the user 810 can be calculated. The viewership score can be used to provide a ranking of the user 810 on a social media platform. In such an embodiment, users that provide more engaging and more frequently viewed content receive higher ratings.

The human face provides a powerful communications medium through its ability to exhibit a myriad of expressions that can be captured and analyzed for a variety of purposes. In some cases, media producers are acutely interested in evaluating the effectiveness of message delivery by video media. Such video media includes advertisements, political messages, educational materials, television programs, movies, government service announcements, etc. Automated facial analysis can be performed on one or more video frames containing a face in order to detect facial action. Based on the facial action detected, a variety of parameters can be determined, including affect valence, spontaneous reactions, facial action units, and so on. The parameters that are determined can be used to infer or predict emotional and mental states. For example, determined valence can be used to describe the emotional reaction of a viewer to a video media presentation or another type of presentation. Positive valence provides evidence that a viewer is experiencing a favorable emotional response to the video media presentation, while negative valence provides evidence that a viewer is experiencing an unfavorable emotional response to the video media presentation. Other facial data analysis can include the determination of discrete emotional states of the viewer or viewers.

Facial data can be collected from a plurality of people using any of a variety of cameras. A camera can include a webcam, a video camera, a still camera, a thermal imager, a CCD device, a smartphone camera, a three-dimensional camera, a depth camera, a light field camera, multiple webcams used to show different views of a person, or any other type of image capture apparatus that can allow captured data to be used in an electronic system. In some embodiments, the person is permitted to “opt-in” to the facial data collection. For example, the person can agree to the capture of facial data using a personal device such as a mobile device or another electronic device by selecting an opt-in choice. Opting-in can then turn on the person's webcam-enabled device and can begin the capture of the person's facial data via a video feed from the webcam or other camera. The video data that is collected can include one or more persons experiencing an event. The one or more persons can be sharing a personal electronic device or can each be using one or more devices for video capture. The videos that are collected can be collected using a web-based framework. The web-based framework can be used to display the video media presentation or event as well as to collect videos from multiple viewers who are online. That is, the collection of videos can be crowdsourced from those viewers who elected to opt-in to the video data collection.

The videos captured from the various viewers who chose to opt-in can be substantially different in terms of video quality, frame rate, etc. As a result, the facial video data can be scaled, rotated, and otherwise adjusted to improve consistency. Human factors further play into the capture of the facial video data. The facial data that is captured might or might not be relevant to the video media presentation being displayed. For example, the viewer might not be paying attention, might be fidgeting, might be distracted by an outside object or event near the viewer, or otherwise inattentive to the video media presentation. The behavior exhibited by the viewer can prove challenging to analyze due to viewer actions including eating, interacting with another person or persons, speaking on the phone, etc. The videos collected from the viewers might also include other artifacts that pose challenges during the analysis of the video data. The artifacts can include items such as eyeglasses (because of reflections), eye patches, jewelry, and clothing that occludes or obscures the viewer's face. Similarly, a viewer's hair or hair covering can present artifacts by obscuring the viewer's eyes and/or face.

The captured facial data can be analyzed using the facial action coding system (FACS). The FACS seeks to define groups or taxonomies of facial movements of the human face. The FACS encodes movements of individual muscles of the face, where the muscle movements often include slight, instantaneous changes in facial appearance. The FACS encoding is commonly performed by trained observers but can also be performed on automated, computer-based systems. Analysis of the FACS encoding can be used to determine emotions of the persons whose facial data is captured in the videos. The FACS is used to encode a wide range of facial expressions that are anatomically possible for the human face. The FACS encodings include action units (AUs) and related temporal segments that are based on the captured facial expression. The AUs are open to higher order interpretation and decision-making. These AUs can be used to recognize emotions experienced by the observed person. Emotion-related facial actions can be identified using the emotional facial action coding system (EMFACS) and the facial action coding system affect interpretation dictionary (FACSAID). For a given emotion, specific action units can be related to the emotion. For example, the emotion of anger can be related to AUs 4, 5, 7, and 23, while happiness can be related to AUs 6 and 12. Other mappings of emotions to AUs have also been previously associated. The coding of the AUs can include an intensity scoring that ranges from A (trace) to E (maximum). The AUs can be used for analyzing images to identify patterns indicative of a particular mental and/or emotional state. The AUs range in number from 0 (neutral face) to 98 (fast up-down look). The AUs include so-called main codes (inner brow raiser, lid tightener, etc.), head movement codes (head turn left, head up, etc.), eye movement codes (eyes turned left, eyes up, etc.), visibility codes (eyes not visible, entire face not visible, etc.), and gross behavior codes (sniff, swallow, etc.). Emotion scoring can be included where intensity is evaluated, as well as specific emotions, moods, or mental states.

The coding of faces identified in videos captured of people observing an event can be automated. The automated systems can detect facial AUs or discrete emotional states. The emotional states can include amusement, fear, anger, disgust, surprise, and sadness. The automated systems can be based on a probability estimate from one or more classifiers, where the probabilities can correlate with an intensity of an AU or an expression. The classifiers can be used to identify into which of a set of categories a given observation can be placed. In some cases, the classifiers can be used to determine a probability that a given AU or expression is present in a given frame of a video. The classifiers can be used as part of a supervised machine learning technique, where the machine learning technique can be trained using “known good” data. Once trained, the machine learning technique can proceed to classify new data that is captured.

The supervised machine learning models can be based on support vector machines (SVMs). An SVM can have an associated learning model that is used for data analysis and pattern analysis. For example, an SVM can be used to classify data that can be obtained from collected videos of people experiencing a media presentation. An SVM can be trained using “known good” data that is labeled as belonging to one of two categories (e.g. smile and no-smile). The SVM can build a model that assigns new data into one of the two categories. The SVM can construct one or more hyperplanes that can be used for classification. The hyperplane that has the largest distance from the nearest training point can be determined to have the best separation. The largest separation can improve the classification technique by increasing the probability that a given data point can be properly classified.

In another example, a histogram of oriented gradients (HoG) can be computed. The HoG can include feature descriptors and can be computed for one or more facial regions of interest. The regions of interest of the face can be located using facial landmark points, where the facial landmark points can include outer edges of nostrils, outer edges of the mouth, outer edges of eyes, etc. A HoG for a given region of interest can count occurrences of gradient orientation within a given section of a frame from a video, for example. The gradients can be intensity gradients and can be used to describe an appearance and a shape of a local object. The HoG descriptors can be determined by dividing an image into small, connected regions, also called cells. A histogram of gradient directions or edge orientations can be computed for pixels in the cell. Histograms can be contrast-normalized based on intensity across a portion of the image or the entire image, thus reducing any influence from illumination or shadowing changes between and among video frames. The HoG can be computed on the image or on an adjusted version of the image, where the adjustment of the image can include scaling, rotation, etc. The image can be adjusted by flipping the image around a vertical line through the middle of a face in the image. The symmetry plane of the image can be determined from the tracker points and landmarks of the image.

In embodiments, an automated facial analysis system identifies five facial actions or action combinations in order to detect spontaneous facial expressions for media research purposes. Based on the facial expressions that are detected, a determination can be made with regard to the impact a given video media presentation has on the viewer, for example. The system can detect the presence of the AUs or the combination of AUs in videos collected from a plurality of people. The facial analysis technique can be trained using a web-based framework to crowdsource videos of people as they watch online video content. The video can be streamed at a fixed frame rate to a server. Human labelers can code for the presence or absence of facial actions including a symmetric smile, unilateral smile, asymmetric smile, and so on. The trained system can then be used to automatically code the facial data collected from a plurality of viewers experiencing video presentations (e.g. television programs).

Spontaneous asymmetric smiles can be detected in order to understand viewer experiences. Related literature indicates that as many asymmetric smiles occur on the right hemi face as do on the left hemi face, for spontaneous expressions. Detection can be treated as a binary classification problem, where images that contain a right asymmetric expression are used as positive (target class) samples and all other images as negative (non-target class) samples. Classifiers perform the classification, including classifiers such as support vector machines (SVM) and random forests. Random forests can include ensemble-learning methods that use multiple learning algorithms to obtain better predictive performance. Frame-by-frame detection can be performed to recognize the presence of an asymmetric expression in each frame of a video. Facial points can be detected, including the top of the mouth and the two outer eye corners. The face can be extracted, cropped, and warped into a pixel image of specific dimension (e.g. 96×96 pixels). In embodiments, the inter-ocular distance and vertical scale in the pixel image are fixed. Feature extraction can be performed using computer vision software such as OpenCV™. Feature extraction can be based on the use of HoGs. HoGs can include feature descriptors and can be used to count occurrences of gradient orientation in localized portions or regions of the image. Other techniques can be used for counting occurrences of gradient orientation, including edge orientation histograms, scale-invariant feature transformation descriptors, etc. The AU recognition tasks can also be performed using Local Binary Patterns (LBP) and Local Gabor Binary Patterns (LGBP). The HoG descriptor represents the face as a distribution of intensity gradients and edge directions, and is robust in its ability to translate and scale. Differing patterns, including groupings of cells of various sizes and arranged in variously sized cell blocks, can be used. For example, 4×4 cell blocks of 8×8 pixel cells with an overlap of half of the block can be used. Histograms of channels can be used, including nine channels or bins evenly spread over 0-180 degrees. In this example, the HoG descriptor on a 96×96 image is 25 blocks×16 cells×9 bins=3600, the latter quantity representing the dimension. AU occurrences can be rendered. The videos can be grouped into demographic datasets based on nationality and/or other demographic parameters for further detailed analysis. This grouping and other analyses can be facilitated via semiconductor-based logic.

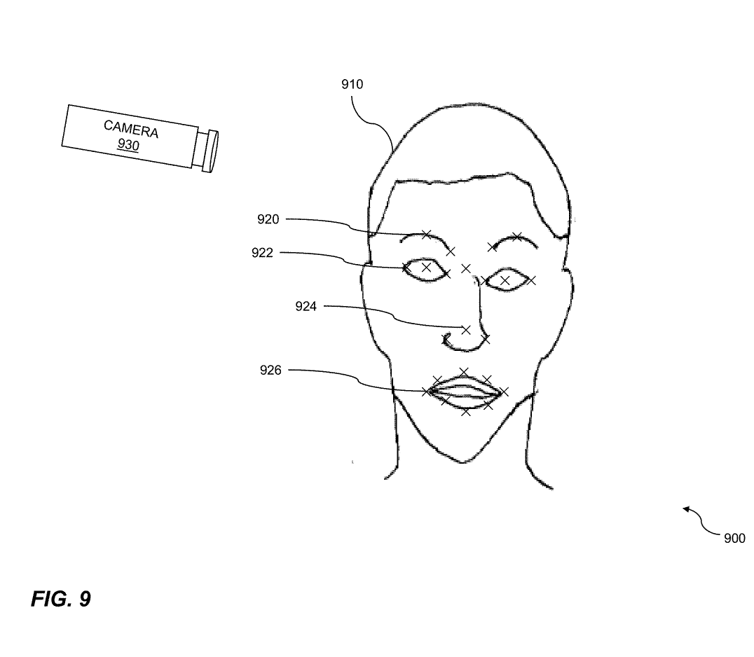

FIG. 9 shows example facial data collection including landmarks. The landmarks can be evaluated within tagging and mood analysis of mental state data collected from multiple sources. The collecting of facial data including landmarks can be performed for images that have been collected of an individual. The collected images can be analyzed for mental states and/or facial expressions. A plurality of images of an individual viewing an electronic display can be received. A face can be identified in an image, based on the use of classifiers. The plurality of images can be evaluated to determine mental states and/or facial expressions of the individual. In the example 900, facial data including facial landmarks can be collected using a variety of electronic hardware and software techniques. The collecting of facial data including landmarks can be based on sub-sectional components of a population. The sub-sectional components can be used with performing the evaluation of content of the face, identifying facial landmarks, etc. The sub-sectional components can be used to provide a context. A face 910 can be observed using a camera 930 in order to collect facial data that includes facial landmarks. The facial data can be collected from a plurality of people using one or more of a variety of cameras. As previously discussed, the camera or cameras can include a webcam, where a webcam can include a video camera, a still camera, a thermal imager, a CCD device, a smartphone camera, a three-dimensional camera, a depth camera, a light field camera, multiple webcams used to show different views of a person, or any other type of image capture apparatus that can allow captured data to be used in an electronic system. The quality and usefulness of the facial data that is captured can depend on the position of the camera 930 relative to the face 910, the number of cameras used, the illumination of the face, etc. In some cases, if the face 910 is poorly lit or over-exposed (e.g. in an area of bright light), the processing of the facial data to identify facial landmarks might be rendered more difficult. In another example, the camera 930 being positioned to the side of the person might prevent capture of the full face. Artifacts can degrade the capture of facial data. For example, the person's hair, prosthetic devices (e.g. glasses, an eye patch, and eye coverings), jewelry, and clothing can partially or completely occlude or obscure the person's face. Data relating to various facial landmarks can include a variety of facial features. The facial features can comprise an eyebrow 920, an outer eye edge 922, a nose 924, a corner of a mouth 926, and so on. Multiple facial landmarks can be identified from the facial data that is captured. The facial landmarks that are identified can be analyzed to identify facial action units. The action units that can be identified can include AU02 outer brow raiser, AU14 dimpler, AU17 chin raiser, and so on. Multiple action units can be identified. The action units can be used alone and/or in combination to infer one or more mental states and emotions. A similar process can be applied to gesture analysis (e.g. hand gestures) with all of the analysis being accomplished or augmented by a mobile device, a server, semiconductor-based logic, and so on.

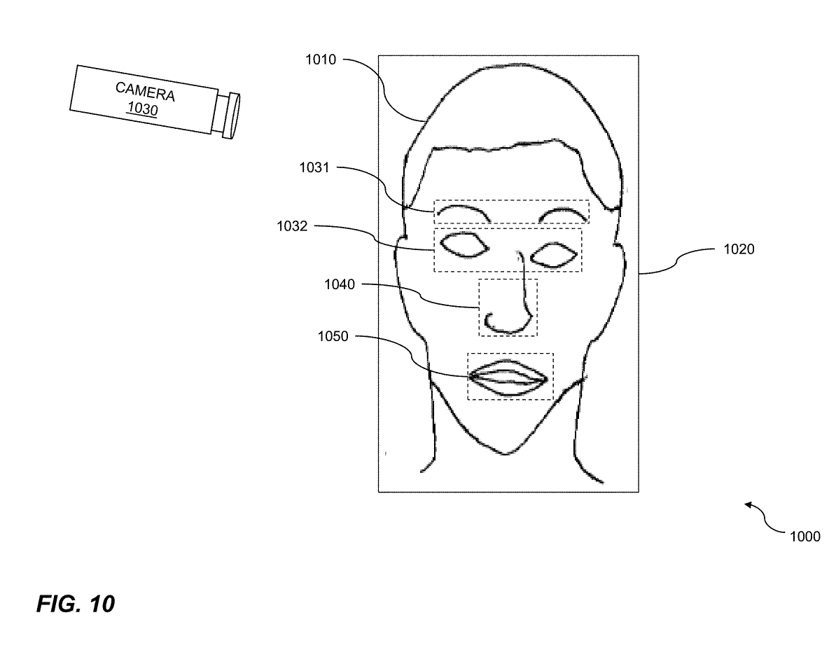

FIG. 10 shows example facial data collection including regions. The regions can be evaluated within tagging and mood analysis of mental state data collected from multiple sources. The collecting of facial data including regions can be performed for images captured of an individual. The captured images can be analyzed for mental states and/or facial expressions. A plurality of images of an individual viewing an electronic display can be received. A face can be identified in an image, based on the use of classifiers. The plurality of images can be evaluated to determine mental states and/or facial expressions of the individual. Various regions of a face can be identified and used for a variety of purposes including facial recognition, facial analysis, and so on. The collecting of facial data including regions can be based on sub-sectional components of a population. The sub-sectional components can be used with performing the evaluation of content of the face, identifying facial regions, etc. The sub-sectional components can be used to provide a context. Facial analysis can be used to determine, predict, and estimate mental states and emotions of a person from whom facial data can be collected.

In embodiments, the one or more emotions that can be determined by the analysis can be represented by an image, a figure, an icon, etc. The representative icon can include an emoji or emoticon. One or more emoji can be used to represent a mental state, emotion, or mood of an individual; to represent food, a geographic location, weather, and so on. The emoji can include a static image. The static image can be a predefined size such as a certain number of pixels. The emoji can include an animated image. The emoji can be based on a GIF or another animation standard. The emoji can include a cartoon representation. The cartoon representation can be any cartoon type, format, etc. that can be appropriate to representing an emoji. In the example 1000, facial data can be collected, where the facial data can include regions of a face. The facial data that is collected can be based on sub-sectional components of a population. When more than one face can be detected in an image, facial data can be collected for one face, some faces, all faces, and so on. The facial data which can include facial regions can be collected using any of a variety of electronic hardware and software techniques. The facial data can be collected using sensors, including motion sensors, infrared sensors, physiological sensors, imaging sensors, and so on. A face 1010 can be observed using a camera 1030, a sensor, a combination of cameras and/or sensors, and so on. The camera 1030 can be used to collect facial data that can be used to determine that a face is present in an image. When a face is present in an image, a bounding box 1020 can be placed around the face. Placement of the bounding box around the face can be based on detection of facial landmarks. The camera 1030 can be used to collect facial data from the bounding box 1020, where the facial data can include facial regions. The facial data can be collected from a plurality of people using any of a variety of cameras. As discussed previously, the camera or cameras can include a webcam, where a webcam can include a video camera, a still camera, a thermal imager, a CCD device, a smartphone camera, a three-dimensional camera, a depth camera, a light field camera, multiple webcams used to show different views of a person, or any other type of image capture apparatus that can allow captured data to be used in an electronic system. As discussed previously, the quality and usefulness of the facial data that is captured can depend on, among other examples, the position of the camera 1030 relative to the face 1010, the number of cameras and/or sensors used, the illumination of the face, any obstructions to viewing the face, and so on.

The facial regions that can be collected by the camera 1030, a sensor, or a combination of cameras and/or sensors can include any of a variety of facial features. Embodiments include determining regions within the face of the individual and evaluating the regions for emotional content. The facial features that can be included in the facial regions that are collected can include eyebrows 1031, eyes 1032, a nose 1040, a mouth 1050, ears, hair, texture, tone, and so on. Multiple facial features can be included in one or more facial regions. The number of facial features that can be included in the facial regions can depend on the desired amount of data to be captured, whether a face is in profile, whether the face is partially occluded or obstructed, etc. The facial regions that can include one or more facial features can be analyzed to determine facial expressions. The analysis of the facial regions can also include determining probabilities of occurrence of one or more facial expressions. The facial features that can be analyzed can also include features such as textures, gradients, colors, and shapes. The facial features can be used to determine demographic data, where the demographic data can include age, ethnicity, culture, and gender. Multiple textures, gradients, colors, shapes, and so on, can be detected by the camera 1030, a sensor, or a combination of cameras and sensors. Texture, brightness, contrast, and color, for example, can be used to detect boundaries in an image for detection of a face, facial features, facial landmarks, and so on.

A texture in a facial region can include facial characteristics, skin types, and so on. In some instances, a texture in a facial region can include smile lines, crow's feet, and wrinkles, among others. Another texture that can be used to evaluate a facial region can include a smooth portion of skin such as a smooth portion of a cheek. A gradient in a facial region can include values assigned to local skin texture, shading, etc. A gradient can be used to encode a texture by computing magnitudes in a local neighborhood or portion of an image. The computed values can be compared to discrimination levels, threshold values, and so on. The gradient can be used to determine gender, facial expression, etc. A color in a facial region can include eye color, skin color, hair color, and so on. A color can be used to determine demographic data, where the demographic data can include ethnicity, culture, age, and gender. A shape in a facial region can include the shape of a face, eyes, nose, mouth, ears, and so on. As with color in a facial region, shape in a facial region can be used to determine demographic data including ethnicity, culture, age, gender, and so on.

The facial regions can be detected based on detection of edges, boundaries, and so on, of features that can be included in an image. The detection can be based on various types of analysis of the image. The features that can be included in the image can include one or more faces. A boundary can refer to a contour in an image plane, where the contour can represent ownership of a particular picture element (pixel) from one object, feature, etc. in the image, to another object, feature, and so on, in the image. An edge can be a distinct, low-level change of one or more features in an image. That is, an edge can be detected based on a change, including an abrupt change such as in color or brightness (contrast) within an image. In embodiments, image classifiers are used for the analysis. The image classifiers can include algorithms, heuristics, and so on, and can be implemented using functions, classes, subroutines, code segments, etc. The classifiers can be used to detect facial regions, facial features, and so on. As discussed above, the classifiers can be used to detect textures, gradients, color, shapes, and edges, among others. Any classifier can be used for the analysis, including, but not limited to, density estimation, support vector machines (SVM), logistic regression, classification trees, and so on. By way of example, consider facial features that can include the eyebrows 1031. One or more classifiers can be used to analyze the facial regions that can include the eyebrows to determine a probability for either a presence or an absence of an eyebrow furrow. The probability can include a posterior probability, a conditional probability, and so on. The probabilities can be based on Bayesian Statistics or other statistical analysis technique. The presence of an eyebrow furrow can indicate the person from whom the facial data was collected is annoyed, confused, unhappy, and so on. In another example, consider facial features that can include a mouth 1050. One or more classifiers can be used to analyze the facial region that can include the mouth to determine a probability for either a presence or an absence of mouth edges turned up to form a smile. Multiple classifiers can be used to determine one or more facial expressions.

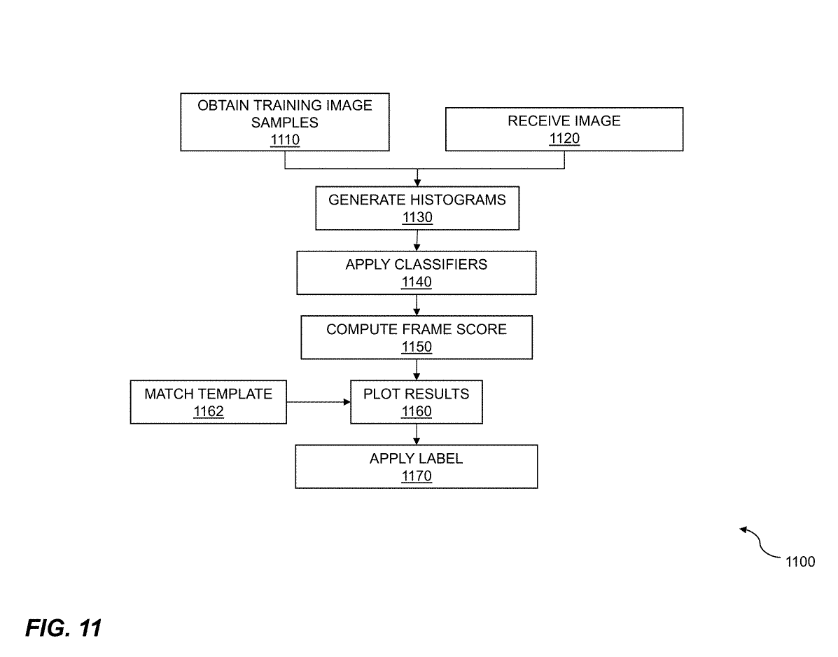

FIG. 11 is a flow diagram for detecting facial expressions. The detection of facial expressions can be performed for data collected from images of an individual and used within tagging and mood analysis of mental state data collected from multiple sources. The collected images can be analyzed for mental states and/or facial expressions. A plurality of images can be received of an individual viewing an electronic display. A face can be identified in an image, based on the use of classifiers. The plurality of images can be evaluated to determine the mental states and/or facial expressions the individual. The flow 1100, or portions thereof, can be implemented in semiconductor-based logic, can be accomplished using a mobile device, can be accomplished using a server device, and so on. The flow 1100 can be used to automatically detect a wide range of facial expressions. A facial expression can produce strong emotional signals that can indicate valence and discrete emotional states. The discrete emotional states can include contempt, doubt, defiance, happiness, fear, anxiety, and so on. The detection of facial expressions can be based on the location of facial landmarks. The detection of facial expressions can be based on determination of action units (AU), where the action units are determined using FACS coding. The AUs can be used singly or in combination to identify facial expressions. Based on the facial landmarks, one or more AUs can be identified by number and intensity. For example, AU12 can be used to code a lip corner puller and can be used to infer a smirk.

The flow 1100 begins by obtaining training image samples 1110. The image samples can include a plurality of images of one or more people. Human coders who are trained to correctly identify AU codes based on the FACS can code the images. The training, or “known good,” images can be used as a basis for training a machine learning technique. Once trained, the machine learning technique can be used to identify AUs in other images that can be collected using a camera, a sensor, and so on. The flow 1100 continues with receiving an image 1120. The image 1120 can be received from a camera, a sensor, and so on. As previously discussed, the camera or cameras can include a webcam, where a webcam can include a video camera, a still camera, a thermal imager, a CCD device, a smartphone camera, a three-dimensional camera, a depth camera, a light field camera, multiple webcams used to show different views of a person, or any other type of image capture apparatus that can allow captured data to be used in an electronic system. The image that is received can be manipulated in order to improve the processing of the image. For example, the image can be cropped, scaled, stretched, rotated, flipped, etc. in order to obtain a resulting image that can be analyzed more efficiently or accurately. Multiple versions of the same image can be analyzed. In some cases, the manipulated image and a flipped or mirrored version of the manipulated image can be analyzed alone and/or in combination to improve analysis. The flow 1100 continues with generating histograms 1130 for the training images and the one or more versions of the received image. The histograms can be based on a HoG or another histogram. As described in previous paragraphs, the HoG can include feature descriptors and can be computed for one or more regions of interest in the training images and the one or more received images. The regions of interest in the images can be located using facial landmark points, where the facial landmark points can include outer edges of nostrils, outer edges of the mouth, outer edges of eyes, etc. A HoG for a given region of interest can count occurrences of gradient orientation within a given section of a frame from a video.

The flow 1100 continues with applying classifiers 1140 to the histograms. The classifiers can be used to estimate probabilities, where the probabilities can correlate with an intensity of an AU or an expression. In some embodiments, the choice of classifiers used is based on the training of a supervised learning technique to identify facial expressions. The classifiers can be used to identify into which of a set of categories a given observation belongs. The classifiers can be used to determine a probability that a given AU or expression is present in a given image or frame of a video. In various embodiments, the one or more AUs that are present include AU01 inner brow raiser, AU12 lip corner puller, AU38 nostril dilator, and so on. In practice, the presence or absence of multiple AUs can be determined. The flow 1100 continues with computing a frame score 1150. The score computed for an image, where the image can be a frame from a video, can be used to determine the presence of a facial expression in the image or video frame. The score can be based on one or more versions of the image 1120 or a manipulated image. The score can be based on a comparison of the manipulated image to a flipped or mirrored version of the manipulated image. The score can be used to predict a likelihood that one or more facial expressions are present in the image. The likelihood can be based on computing a difference between the outputs of a classifier used on the manipulated image and on the flipped or mirrored image, for example. The classifier that is used can be used to identify symmetrical facial expressions (e.g. smile), asymmetrical facial expressions (e.g. outer brow raiser), and so on.

The flow 1100 continues with plotting results 1160. The results that are plotted can include one or more scores for one or more frames computed over a given time t. For example, the plotted results can include classifier probability results from analysis of HoGs for a sequence of images and video frames. The plotted results can be matched with a template 1162. The template can be temporal and can be represented by a centered box function or another function. A best fit with one or more templates can be found by computing a minimum error. Other best-fit techniques can include polynomial curve fitting, geometric curve fitting, and so on. The flow 1100 continues with applying a label 1170. The label can be used to indicate that a particular facial expression has been detected in the one or more images or video frames which constitute the image 1120 that was received. The label can be used to indicate that any of a range of facial expressions has been detected, including a smile, an asymmetric smile, a frown, and so on. Various steps in the flow 1100 may be changed in order, repeated, omitted, or the like without departing from the disclosed concepts. Various embodiments of the flow 1100 can be included in a computer program product embodied in a non-transitory computer readable medium that includes code executable by one or more processors. Various embodiments of the flow 1100, or portions thereof, can be included on a semiconductor chip and implemented in special purpose logic, programmable logic, and so on.

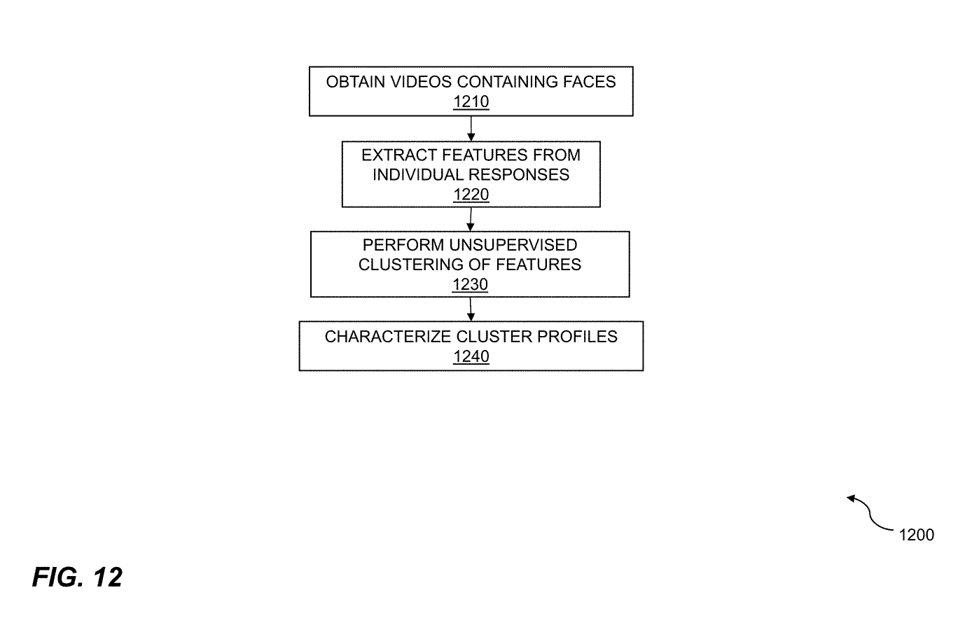

FIG. 12 is a flow diagram for the large-scale clustering of facial events. The large-scale clustering of facial events can be performed for data collected from images of an individual and used within tagging and mood analysis of mental state data collected from multiple sources. The collected images can be analyzed for mental states and/or facial expressions. A plurality of images can be received of an individual viewing an electronic display. A face can be identified in an image, based on the use of classifiers. The plurality of images can be evaluated to determine the mental states and/or facial expressions of the individual. The clustering and evaluation of facial events can be augmented using a mobile device, a server, semiconductor-based logic, and so on. As discussed above, collection of facial video data from one or more people can include a web-based framework. The web-based framework can be used to collect facial video data from large numbers of people located over a wide geographic area. The web-based framework can include an opt-in feature that allows people to agree to facial data collection. The web-based framework can be used to render and display data to one or more people and can collect data from the one or more people. For example, the facial data collection can be based on showing one or more viewers a video media presentation through a website. The web-based framework can be used to display the video media presentation or event and to collect videos from multiple viewers who are online. That is, the collection of videos can be crowdsourced from those viewers who elected to opt-in to the video data collection. The video event can be a commercial, a political ad, an educational segment, and so on.

The flow 1200 begins with obtaining videos containing faces 1210. The videos can be obtained using one or more cameras, where the cameras can include a webcam coupled to one or more devices employed by the one or more people using the web-based framework. The flow 1200 continues with extracting features from the individual responses 1220. The individual responses can include videos containing faces observed by the one or more webcams. The features that are extracted can include facial features such as an eyebrow, a nostril, an eye edge, a mouth edge, and so on. The feature extraction can be based on facial coding classifiers, where the facial coding classifiers output a probability that a specified facial action has been detected in a given video frame. The flow 1200 continues with performing unsupervised clustering of features 1230. The unsupervised clustering can be based on an event. The unsupervised clustering can be based on a K-Means, where the K of the K-Means can be computed using a Bayesian Information Criterion (BICk), for example, to determine the smallest value of K that meets system requirements. Any other criterion for K can be used. The K-Means clustering technique can be used to group one or more events into various respective categories.

The flow 1200 continues with characterizing cluster profiles 1240. The profiles can include a variety of facial expressions such as smiles, asymmetric smiles, eyebrow raisers, eyebrow lowerers, etc. The profiles can be related to a given event. For example, a humorous video can be displayed in the web-based framework and the video data of people who have opted-in can be collected. The characterization of the collected and analyzed video can depend in part on the number of smiles that occurred at various points throughout the humorous video. The number of smiles resulting from people viewing a humorous video can be compared to various demographic groups, where the groups can be formed based on geographic location, age, ethnicity, gender, and so on. Similarly, the characterization can be performed on collected and analyzed videos of people viewing a news presentation. The characterized cluster profiles can be further analyzed based on demographic data. Various steps in the flow 1200 may be changed in order, repeated, omitted, or the like without departing from the disclosed concepts. Various embodiments of the flow 1200 can be included in a computer program product embodied in a non-transitory computer readable medium that includes code executable by one or more processors. Various embodiments of the flow 1200, or portions thereof, can be included on a semiconductor chip and implemented in special purpose logic, programmable logic, and so on.

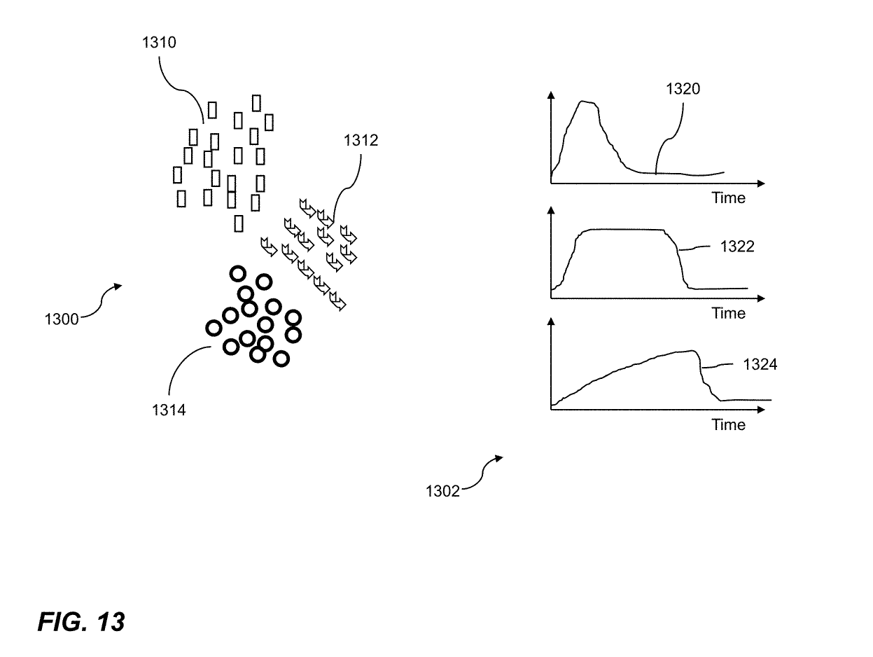

FIG. 13 shows unsupervised clustering of features and characterizations of cluster profiles. The clustering can be accomplished within tagging and mood analysis of mental state data collected from multiple sources. The clustering of features and characterizations of cluster profiles can be performed for images collected of an individual. The collected images can be analyzed for mental states and/or facial expressions. A plurality of images can be received of an individual viewing an electronic display. A face can be identified in an image, based on the use of classifiers. The plurality of images can be evaluated to determine mental states and/or facial expressions of the individual. Features including samples of facial data can be clustered using unsupervised clustering. Various clusters can be formed which include similar groupings of facial data observations. The example 1300 shows three clusters, clusters 1310, 1312, and 1314. The clusters can be based on video collected from people who have opted-in to video collection. When the data collected is captured using a web-based framework, the data collection can be performed on a grand scale, including hundreds, thousands, or even more participants who can be located locally and/or across a wide geographic area. Unsupervised clustering is a technique that can be used to process the large amounts of captured facial data and to identify groupings of similar observations among participants. The unsupervised clustering can also be used to characterize the groups of similar observations. The characterizations can include identifying behaviors of the participants. The characterizations can be based on identifying facial expressions and facial action units of the participants. Some behaviors and facial expressions can include faster or slower onsets, faster or slower offsets, longer or shorter durations, etc. The onsets, offsets, and durations can all correlate to time. The data clustering that results from the unsupervised clustering can support data labeling. The labeling can include FACS coding. The clusters can be partially or totally based on a facial expression resulting from participants viewing a video presentation, where the video presentation can be an advertisement, a political message, educational material, a public service announcement, and so on. The clusters can be correlated with demographic information, where the demographic information can include educational level, geographic location, age, gender, income level, and so on.

The cluster profiles 1302 can be generated based on the clusters that can be formed from unsupervised clustering, with time shown on the x-axis and intensity or frequency shown on the y-axis. The cluster profiles can be based on captured facial data including facial expressions. The cluster profile 1320 can be based on the cluster 1310, the cluster profile 1322 can be based on the cluster 1312, and the cluster profile 1324 can be based on the cluster 1314. The cluster profiles 1320, 1322, and 1324 can be based on smiles, smirks, frowns, or any other facial expression. The emotional states of the people who have opted-in to video collection can be inferred by analyzing the clustered facial expression data. The cluster profiles can be plotted with respect to time and can show a rate of onset, a duration, and an offset (rate of decay). Other time-related factors can be included in the cluster profiles. The cluster profiles can be correlated with demographic information, as described above.

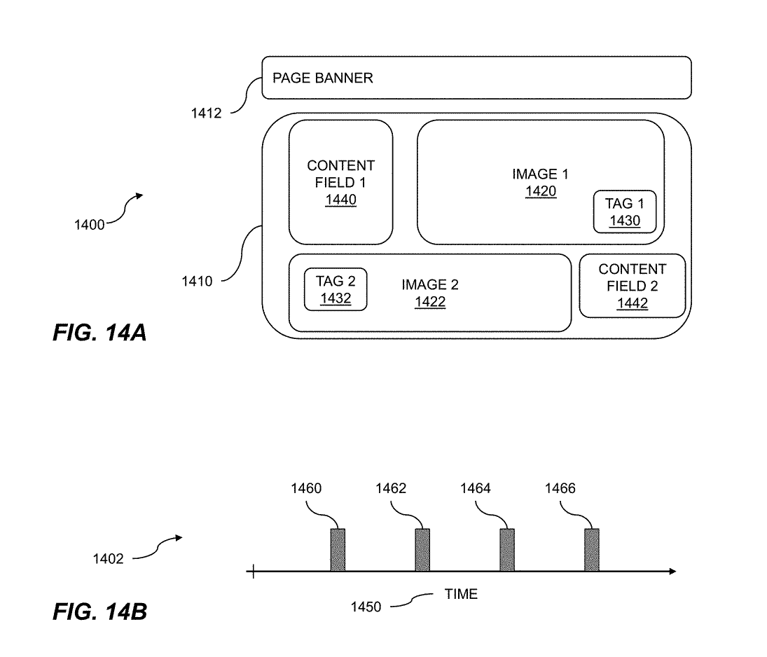

FIG. 14A shows example tags embedded in a webpage. The tags embedded in the webpage can be used for image analysis for images collected of an individual, and the image analysis can be performed within tagging and mood analysis of mental state data collected from multiple sources. The collected images can be analyzed for mental states and/or facial expressions. A plurality of images can be received of an individual viewing an electronic display. A face can be identified in an image, based on the use of classifiers. The plurality of images can be evaluated to determine mental states and/or facial expressions of the individual. Once a tag is detected, a mobile device, a server, semiconductor-based logic, etc. can be used to evaluate associated facial expressions. A webpage 1400 can include a page body 1410, a page banner 1412, and so on. The page body can include one or more objects, where the objects can include text, images, videos, audio, and so on. The example page body 1410 shown includes a first image, image 1 1420; a second image, image 2 1422; a first content field, content field 1 1440; and a second content field, content field 2 1442. In practice, the page body 1410 can contain multiple images and content fields, and can include one or more videos, one or more audio presentations, and so on. The page body can include embedded tags, such as tag 1 1430 and tag 2 1432. In the example shown, tag 1 1430 is embedded in image 1 1420, and tag 2 1432 is embedded in image 2 1422. In embodiments, multiple tags are imbedded. Tags can also be imbedded in content fields, in videos, in audio presentations, etc. When a user mouses over a tag or clicks on an object associated with a tag, the tag can be invoked. For example, when the user mouses over tag 1 1430, tag 1 1430 can then be invoked. Invoking tag 1 1430 can include enabling a camera coupled to a user's device and capturing one or more images of the user as the user views a media presentation or other digital experience. In a similar manner, when the user mouses over tag 2 1432, tag 2 1432 can be invoked. Invoking tag 2 1432 can also include enabling the camera and capturing images of the user. In other embodiments, other actions are taken based on invocation of the one or more tags. Invoking an embedded tag can initiate an analysis technique, post to social media, award the user a coupon or another prize, initiate mental state analysis, perform emotion analysis, and so on.

FIG. 14B shows invoking tags to collect images. The invoking tags to collect images can be used for image analysis for images collected of an individual and used within tagging and mood analysis of mental state data collected from multiple sources. The collected images can be analyzed for mental states and/or facial expressions. A plurality of images can be received of an individual viewing an electronic display. A face can be identified in an image, based on the use of classifiers. The plurality of images can be evaluated to determine mental states and/or facial expressions of the individual. As previously stated, a media presentation can be a video, a webpage, and so on. A video 1402 can include one or more embedded tags, such as a tag 1460, another tag 1462, a third tag 1464, a fourth tag 1466, and so on. In practice, multiple tags can be included in the media presentation. The one or more tags can be invoked during the media presentation. The collection of the invoked tags can occur over time, as represented by a timeline 1450. When a tag is encountered in the media presentation, the tag can be invoked. When the tag 1460 is encountered, invoking the tag can enable a camera coupled to a user device and can capture one or more images of the user viewing the media presentation. Invoking a tag can depend on opt-in by the user. For example, if a user has agreed to participate in a study by indicating an opt-in, then the camera coupled to the user's device can be enabled and one or more images of the user can be captured. If the user has not agreed to participate in the study and has not indicated an opt-in, then invoking the tag 1460 does not enable the camera nor capture images of the user during the media presentation. The user can indicate an opt-in for certain types of participation, where opting-in can be dependent on specific content in the media presentation. The user could opt-in to participation in a study of political campaign messages and not opt-in for a particular advertisement study. In this case, tags that are related to political campaign messages, advertising messages, social media sharing, etc. and that enable the camera and image capture when invoked would be embedded in the media presentation, social media sharing, and so on. However, tags imbedded in the media presentation that are related to advertisements would not enable the camera when invoked. Various other situations of tag invocation are possible.

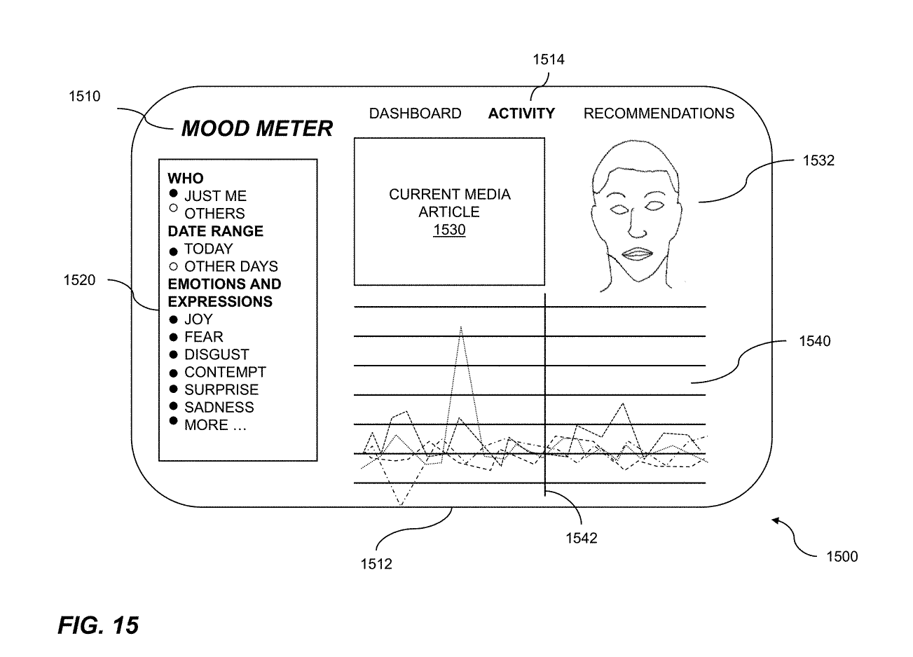

FIG. 15 shows an example mood measurement display for individual activity. A mood measurement for individual activity can be based on various techniques including being based on mental states inferred from heart rate information and/or facial image analysis. Emotional moods of an individual can be analyzed from mental states. Mental state data and/or facial data can be collected on the individual. The collecting of the mental state data can be accomplished using a webcam or other video capture device, or it can be inferred from heart rate information. Processors are used to analyze the mental state data for providing analysis of the mental state data to the individual in the form of a mood. Mental state data may include many sub-states of emotion, but a mood is a prevailing or overriding emotion or an emotion or mental state of interest, such as overall happiness. Mental state data is inferred and outputted as a mood measurement. An example display 1500, such as example mood measurement 1510, shows individual activity 1512. The display can include controls 1514 for selecting among various dashboards, displaying activity, recommending ways for improving a mood, and so on. The dashboard can include activity 1520 of the individual and can include the activity of others, a date range, a list of emotions and expressions, selfie settings, screenshot settings, and so on. The display can include an image of a webpage or current media article 1530 being observed by the individual, along with an image of the individual 1532. The display can include moment-by-moment metrics 1540 tracked as a graph over time, including joy, fear, disgust, contempt, surprise, sadness, etc. The moment-by-moment metrics 1540 can include physiological data that can be captured along with the collecting mental state data. The moment-by-moment metrics 1540 can include an indication of a specific point in time 1542, which can be moved along the time axis.

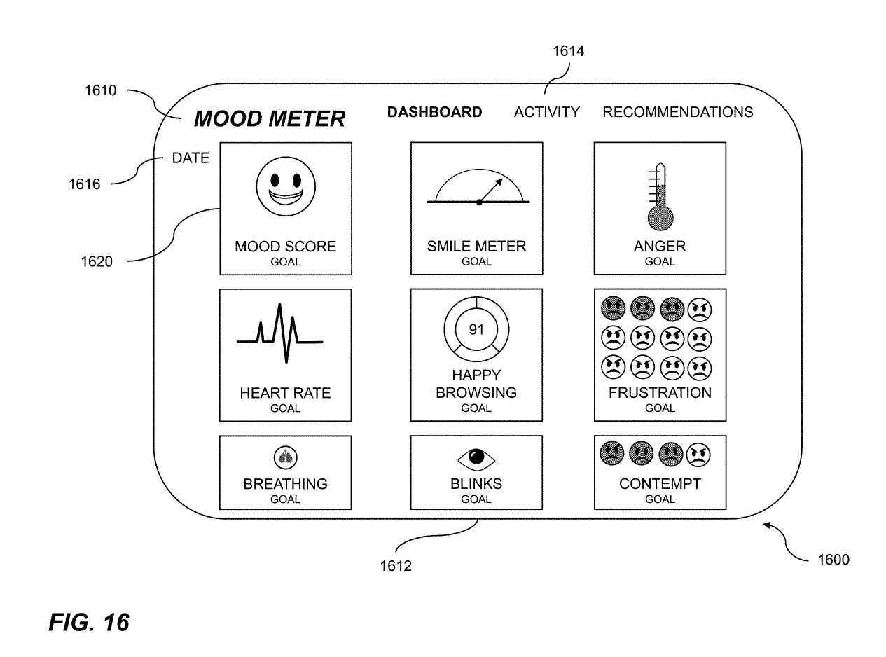

FIG. 16 illustrates an example mood measurement dashboard display. A dashboard can be used to display information to an individual, where the information is based on inferred mental states from heart rate information and/or facial image analysis. Emotional moods of an individual can be analyzed from mental states. Mental state data and/or facial data can be collected on the individual. The collecting of the mental state data can be accomplished using a webcam or other video capture device, or it can be inferred from heart rate information. Processors are used to analyze the mental state data for providing analysis of the mental state data to the individual in the form of a mood. Mental state data may include many sub-states of emotion, but a mood is a prevailing or overriding emotion or an emotion or mental state of interest, such as overall happiness. Mental state data is inferred and outputted as a mood measurement. An example display 1600, such as example mood measurement 1610, can display individual mood dashboard information 1612 to an individual. A variety of information can be displayed including a mood score 1620, a meter, such as a smile meter and a target number of smiles per day, an anger meter with daily goal, a heart rate with daily goal, a browsing mood such as happy browsing with a daily goal, a frustration meter and goal, a breathing meter and goal, an eye blinks meter and goal, a contempt meter and goal, and so on. The dashboard 1612 can include controls 1614 which can be used to select among multiple dashboards, to display various activities, to take action or receive suggestions for such activities as improving a mood, and so on. A selectable date 1616 can be employed to display moods for various days in the past.

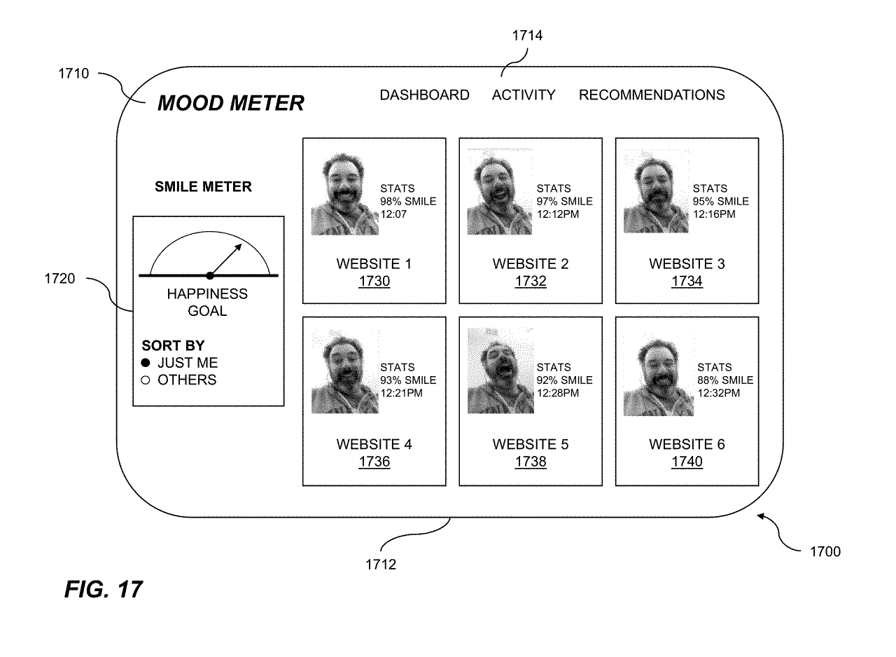

FIG. 17 illustrates example mood measurement statistical results. Various statistical results can be displayed for an individual, where the information is based on inferred mental states from heart rate information and/or facial image analysis. Emotional moods of an individual can be analyzed from mental states. Mental state data and/or facial data can be collected on the individual. The collecting of the mental state data can be accomplished using a webcam or other video capture device, or it can be inferred from heart rate information. Processors are used to analyze the mental state data for providing analysis of the mental state data to the individual in the form of a mood. Mental state data may include many sub-states of emotion, but a mood is a prevailing or overriding emotion or an emotion or mental state of interest, such as overall happiness. Mental state data is inferred and outputted as a mood measurement. Statistical results 1700 based on analyzing and evaluating can be displayed to an individual using a mood measurement display 1710. The display can show statistical results for a variety of moods such as happy, sad, confused, angry, annoyed, concentrating, bored, and so on. The display can show statistical results based on a variety of emotions, mental states, etc. The displayed moods, emotions, mental states, and so on, can be based on aggregating the mental state data from the individual with mental state data from other individuals. The mental state data for the individual can be compared to the aggregated mental state data from the other individuals. The mental state data from other individuals can be based on demographics. The display 1710 can show individual mood statistics 1712. The statistics 1712 can be related to an emotional or mood goal, such as a smile meter/happiness goal 1720. Controls 1714 can be used to select various views, activities, actions, and so on. The display 1710, when displaying smiles, can include a smile meter 1720. The smile meter can include a display for level of happiness, a goal, sorting options such as most recent smile and biggest smiles, selfie settings, screenshot settings, etc. The statistical results of a mood such as a smile can be displayed with various statistics associated with various visited websites or media or games 1730, 1732, 1734, 1736, 1738, and 1740. The statistics can include a percentage of time smiling, the time at which the smile occurred, the website or media or game for which the smile occurred, an image of the individual for whom the statistical results are being displayed, etc. Several renderings of statistics can be display simultaneously.

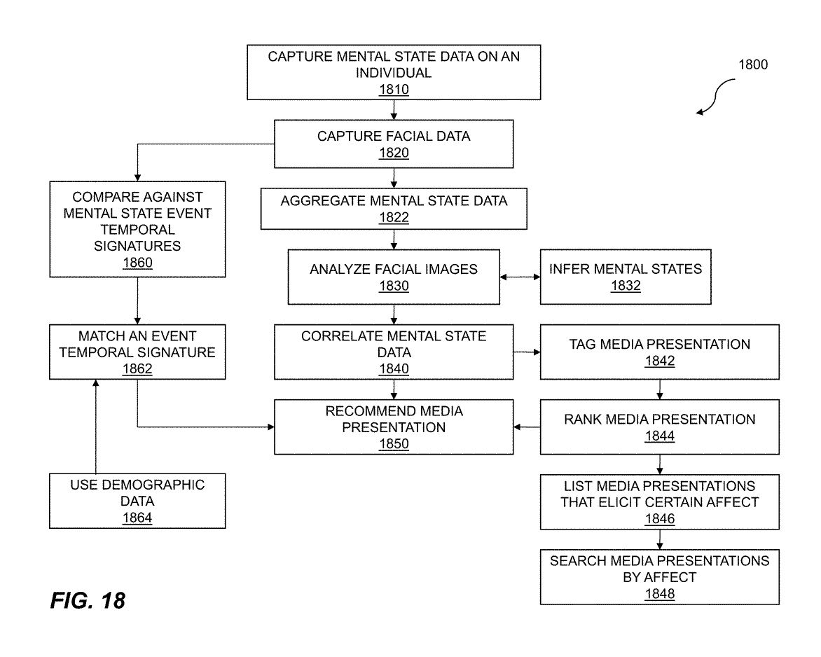

FIG. 18 is a flow diagram for mental state-based recommendations and shows a flow 1800 which describes a computer-implemented method for tagging and mood analysis of mental state data collected from multiple sources. The flow 1800 begins with capturing mental state data on an individual 1810. The capturing can be based on displaying stimuli to an individual or a group of people of which the individual is a part. The displaying can be done all at once or through multiple occurrences. The plurality of media presentations can include videos. The plurality of videos can include YouTube™ videos, Vimeo™ videos, Netflix™ videos, or other video sources. Further, the plurality of media presentations can include a movie, a movie trailer, a television show, a web series, a webisode, a video, a video clip, an advertisement, a music video, an electronic game, an e-book, or an e-magazine. The flow 1800 continues with capturing facial data 1820. The facial data can identify a first face. The captured facial data can be from the individual or from the group of people of which the individual is a part while the plurality of media presentations is displayed. Thus, mental state data can be captured from multiple people. The mental state data can include facial images. In some embodiments, the playing of the media presentations is done on a mobile device and the recording of the facial images is done with the mobile device. The flow 1800 includes aggregating the mental state data 1822 from the multiple people. The flow 1800 further comprises analyzing the facial images 1830 for a facial expression. The facial expression can include a smile or a brow furrow. The flow 1800 can further comprise using the facial images to infer mental states 1832. The mental states can include frustration, confusion, disappointment, hesitation, cognitive overload, focusing, being engaged, attending, boredom, exploration, confidence, trust, delight, valence, skepticism, satisfaction, and the like.

The flow 1800 includes correlating the mental state data 1840 captured from the group of people who have viewed the plurality of media presentations and had their mental state data captured. The plurality of videos viewed by the group of people can have some common videos seen by each of the people in the group of people. In some embodiments, the plurality of videos does not include an identical set of videos. The flow 1800 can continue with tagging the plurality of media presentations 1842 with mental state information based on the mental state data which was captured. In some embodiments, the mental state information can simply be the mental state data, while in other embodiments, the mental state information can be the inferred mental states. In still other embodiments, the mental state information is the results of the correlation. The flow 1800 can continue with ranking the media presentations 1844 relative to another media presentation based on the mental state data which was collected. The ranking can be for an individual based on the mental state data captured from the individual. The ranking can be based on anticipated preferences for the individual. In some embodiments, the ranking of a first media presentation relative to another media presentation is based on the mental state data which was aggregated from multiple people. The ranking can also be relative to media presentations previously stored with mental state information. The ranking can include ranking a video relative to another video based on the mental state data which was captured. The flow 1800 can further comprise displaying the videos which elicit a certain mental state 1846. The certain mental states can include smiles, engagement, attention, interest, sadness, liking, disliking, and so on. The ranking can further comprise displaying the videos which elicited a larger number of smiles. As a result of ranking, the media presentations can be sorted based on which videos are the funniest, the saddest, which generate the most tears, or which engender some other emotional response. The flow 1800 can further comprise searching through the videos based on a certain mental state data 1848. A search 1848 can identify videos which are very engaging, funny, sad, poignant, or the like.

The flow 1800 includes comparing the mental state data that was captured for the individual against a plurality of mental state event temporal signatures 1860. In embodiments, multiple mental state event temporal signatures have been obtained from previous analysis of numerous people. The mental state event temporal signatures can include information on rise time to facial expression intensity, fall time from facial expression intensity, duration of a facial expression, and so on. In some embodiments, the mental state event temporal signatures are associated with certain demographics, ethnicities, cultures, etc. The mental state event temporal signatures can be used to identify one or more of sadness, stress, happiness, anger, frustration, confusion, disappointment, hesitation, cognitive overload, focusing, engagement, attention, boredom, exploration, confidence, trust, delight, disgust, skepticism, doubt, satisfaction, excitement, laughter, calmness, curiosity, humor, depression, envy, sympathy, embarrassment, poignancy, or mirth. The mental state event temporal signatures can be used to identify liking or satisfaction with a media presentation. The mental state event temporal signatures can be used to correlate with appreciating a second media presentation. The flow 1800 can include matching a first event signature 1862, from the plurality of mental state event temporal signatures, against the mental state data that was captured. In embodiments, an output rendering is based on the matching of the first event signature. The matching can include identifying similar aspects of the mental state event temporal signature such as rise time, fall time, duration, and so on. The matching can include matching a series of facial expressions described in mental state event temporal signatures. In some embodiments, a second mental state event temporal signature is used to identify a sequence of mental state data being expressed by an individual. In some embodiments, demographic data 1864 is used to provide a demographic basis for analyzing temporal signatures.

The flow 1800 includes recommending a second media presentation 1850 to an individual based on the mental state data that was captured and based on the ranking. The recommending the second media presentation to the individual is further based on the comparing of the mental state data to the plurality of mental state event temporal signatures. The second media presentation can be a movie, a movie trailer, a television show, a web series, a webisode, a video, a video clip, an advertisement, a music video, an electronic game, an e-book, or an e-magazine. The recommending the second media presentation can be further based on the matching of the first event signature. The recommending can be based on similarity of mental states expressed. The recommending can be based on a numerically quantifiable determination of satisfaction or appreciation of the first media and an anticipated numerically quantifiable satisfaction or appreciation of second first media presentation.

Based on the mental states, recommendations to or from an individual can be provided. One or more recommendations can be made to the individual based on mental states, affect, or facial expressions. A correlation can be made between one individual and others with similar affect exhibited during multiple videos. The correlation can include a record of other videos, games, or other experiences, along with their affect. Likewise, a recommendation for a movie, video, video clip, webisode or another activity can be made to an individual based on their affect. Various steps in the flow 1800 may be changed in order, repeated, omitted, or the like without departing from the disclosed inventive concepts. Various embodiments of the flow 1800 may be included in a computer program product embodied in a non-transitory computer readable medium that includes code executable by one or more processors.

The human face provides a powerful communications medium through its ability to exhibit a myriad of expressions that can be captured and analyzed for a variety of purposes. In some cases, media producers are acutely interested in evaluating the effectiveness of message delivery by video media. Such video media includes advertisements, political messages, educational materials, television programs, movies, government service announcements, etc. Automated facial analysis can be performed on one or more video frames containing a face in order to detect facial action. Based on the facial action detected, a variety of parameters can be determined including affect valence, spontaneous reactions, facial action units, and so on. The parameters that are determined can be used to infer or predict emotional and mental states. For example, determined valence can be used to describe the emotional reaction of a viewer to a video media presentation or another type of presentation. Positive valence provides evidence that a viewer is experiencing a favorable emotional response to the video media presentation, while negative valence provides evidence that a viewer is experiencing an unfavorable emotional response to the video media presentation. Other facial data analysis can include the determination of discrete emotional states of the viewer or viewers.

Facial data can be collected from a plurality of people using any of a variety of cameras. A camera can include a webcam, a video camera, a still camera, a thermal imager, a CCD device, a smartphone camera, a three-dimensional camera, a depth camera, a light field camera, multiple webcams used to show different views of a person, or any other type of image capture apparatus that can allow captured data to be used in an electronic system. In some embodiments, the person is permitted to “opt-in” to the facial data collection. For example, the person can agree to the capture of facial data using a personal device such as a mobile device or another electronic device by selecting an opt-in choice. Opting-in can then turn on the person's webcam-enabled device and can begin the capture of the person's facial data via a video feed from the webcam or other camera. The video data that is collected can include one or more persons experiencing an event. The one or more persons can be sharing a personal electronic device or can each be using one or more devices for video capture. The videos that are collected can be collected using a web-based framework. The web-based framework can be used to display the video media presentation or event as well as to collect videos from any number of viewers who are online. That is, the collection of videos can be crowdsourced from those viewers who elected to opt-in to the video data collection.

In some embodiments, a high frame rate camera is used. A high frame rate camera has a frame rate of 60 frames per second or higher. With such a frame rate, micro expressions can also be captured. Micro expressions are very brief facial expressions, lasting only a fraction of a second. They occur when a person either deliberately or unconsciously conceals a feeling.

In some cases, micro expressions happen when people have hidden their feelings from themselves (repression) or when they deliberately try to conceal their feelings from others. Sometimes the micro expressions might only last about 50 milliseconds. Hence, these expressions can go unnoticed by a human observer. However, a high frame rate camera can be used to capture footage at a sufficient frame rate such that the footage can be analyzed for the presence of micro expressions. Micro expressions can be analyzed via action units as previously described, with various attributes such as brow raising, brow furls, eyelid raising, and the like. Thus, embodiments analyze micro expressions that are easily missed by human observers due to their transient nature.

The videos captured from the various viewers who chose to opt-in can be substantially different in terms of video quality, frame rate, etc. As a result, the facial video data can be scaled, rotated, and otherwise adjusted to improve consistency. Human factors further play into the capture of the facial video data. The facial data that is captured might or might not be relevant to the video media presentation being displayed. For example, the viewer might not be paying attention, might be fidgeting, might be distracted by an object or event near the viewer, or otherwise inattentive to the video media presentation. The behavior exhibited by the viewer can prove challenging to analyze due to viewer actions including eating, interacting with another person or persons nearby, speaking on the phone, etc. The videos collected from the viewers might also include other artifacts that pose challenges during the analysis of the video data. The artifacts can include such items as eyeglasses (because of reflections), eye patches, jewelry, and clothing that occludes or obscures the viewer's face. Similarly, a viewer's hair or hair covering can present artifacts by obscuring the viewer's eyes and/or face.

The captured facial data can be analyzed using the facial action coding system (FACS). The FACS seeks to define groups or taxonomies of facial movements of the human face. The FACS encodes movements of individual muscles of the face, where the muscle movements often include slight, instantaneous changes in facial appearance. The FACS encoding is commonly performed by trained observers, but can also be performed on automated, computer-based systems. Analysis of the FACS encoding can be used to determine emotions of the persons whose facial data is captured in the videos. The FACS is used to encode a wide range of facial expressions that are anatomically possible for the human face. The FACS encodings include action units (AUs) and related temporal segments that are based on the captured facial expression. The AUs are open to higher order interpretation and decision-making. For example, the AUs can be used to recognize emotions experienced by the observed person. Emotion-related facial actions can be identified using the emotional facial action coding system (EMFACS) and the facial action coding system affect interpretation dictionary (FACSAID), for example. For a given emotion, specific action units can be related to the emotion. For example, the emotion of anger can be related to AUs 4, 5, 7, and 23, while happiness can be related to AUs 6 and 12. Other mappings of emotions to AUs have also been previously associated. The coding of the AUs can include an intensity scoring that ranges from A (trace) to E (maximum). The AUs can be used for analyzing images to identify patterns indicative of a particular mental and/or emotional state. The AUs range in number from 0 (neutral face) to 98 (fast up-down look). The AUs include so-called main codes (inner brow raiser, lid tightener, etc.), head movement codes (head turn left, head up, etc.), eye movement codes (eyes turned left, eyes up, etc.), visibility codes (eyes not visible, entire face not visible, etc.), and gross behavior codes (sniff, swallow, etc.). Emotion scoring can be included where intensity is evaluated as well as specific emotions, moods, or mental states.

The coding of faces identified in videos captured of people observing an event can be automated. The automated systems can detect facial AUs or discrete emotional states. The emotional states can include amusement, fear, anger, disgust, surprise, and sadness, for example. The automated systems can be based on a probability estimate from one or more classifiers, where the probabilities can correlate with an intensity of an AU or an expression. The classifiers can be used to identify into which of a set of categories a given observation can be placed. For example, the classifiers can be used to determine a probability that a given AU or expression is present in a given frame of a video. The classifiers can be used as part of a supervised machine learning technique where the machine learning technique can be trained using “known good” data. Once trained, the machine learning technique can proceed to classify new data that is captured.

The supervised machine learning models can be based on support vector machines (SVMs). An SVM can have an associated learning model that is used for data analysis and pattern analysis. For example, an SVM can be used to classify data that can be obtained from collected videos of people experiencing a media presentation. An SVM can be trained using “known good” data that is labeled as belonging to one of two categories (e.g. smile and no-smile). The SVM can build a model that assigns new data into one of the two categories. The SVM can construct one or more hyperplanes that can be used for classification. The hyperplane that has the largest distance from the nearest training point can be determined to have the best separation. The largest separation can improve the classification technique by increasing the probability that a given data point can be properly classified.

In another example, a histogram of oriented gradients (HoG) can be computed. The HoG can include feature descriptors and can be computed for one or more facial regions of interest. The regions of interest of the face can be located using facial landmark points, where the facial landmark points can include outer edges of nostrils, outer edges of the mouth, outer edges of eyes, etc. A HoG for a given region of interest can count occurrences of gradient orientation within a given section of a frame from a video, for example. The gradients can be intensity gradients and can be used to describe an appearance and a shape of a local object. The HoG descriptors can be determined by dividing an image into small, connected regions, also called cells. A histogram of gradient directions or edge orientations can be computed for pixels in the cell. Histograms can be contrast-normalized based on intensity across a portion of the image or the entire image, thus reducing any influence from illumination or shadowing changes between and among video frames. The HoG can be computed on the image or on an adjusted version of the image, where the adjustment of the image can include scaling, rotation, etc. For example, the image can be adjusted by flipping the image around a vertical line through the middle of a face in the image. The symmetry plane of the image can be determined from the tracker points and landmarks of the image.

Embodiments can include identifying a first face and a second face within the facial data. Identifying and analyzing can be accomplished without further interaction with the cloud environment, in coordination with the cloud environment, and so on. In an embodiment, an automated facial analysis system identifies five facial actions or action combinations in order to detect spontaneous facial expressions for media research purposes. Based on the facial expressions that are detected, a determination can be made with regard to the effectiveness of a given video media presentation, for example. The system can detect the presence of the AUs or the combination of AUs in videos collected from a plurality of people. The facial analysis technique can be trained using a web-based framework to crowdsource videos of people as they watch online video content. The video can be streamed at a fixed frame rate to a server. Human labelers can code for the presence or absence of facial actions including symmetric smile, unilateral smile, asymmetric smile, and so on. The trained system can then be used to automatically code the facial data collected from a plurality of viewers experiencing video presentations (e.g. television programs).

Spontaneous asymmetric smiles can be detected in order to understand viewer experiences. Related literature indicates that as many asymmetric smiles occur on the right hemi face as do on the left hemi face, for spontaneous expressions. Detection can be treated as a binary classification problem, where images that contain a right asymmetric expression are used as positive (target class) samples and all other images as negative (non-target class) samples. Classifiers perform the classification, including classifiers such as support vector machines (SVM) and random forests. Random forests can include ensemble-learning methods that use multiple learning algorithms to obtain better predictive performance. Frame-by-frame detection can be performed to recognize the presence of an asymmetric expression in each frame of a video. Facial points can be detected, including the top of the mouth and the two outer eye corners. The face can be extracted, cropped, and warped into a pixel image of specific dimension (e.g. 96×96 pixels). In embodiments, the inter-ocular distance and vertical scale in the pixel image are fixed. Feature extraction can be performed using computer vision software such as OpenCV™. Feature extraction can be based on the use of HoGs. HoGs can include feature descriptors and can be used to count occurrences of gradient orientation in localized portions or regions of the image. Other techniques can be used for counting occurrences of gradient orientation, including edge orientation histograms, scale-invariant feature transformation descriptors, etc. The AU recognition tasks can also be performed using Local Binary Patterns (LBP) and Local Gabor Binary Patterns (LGBP). The HoG descriptor represents the face as a distribution of intensity gradients and edge directions, and is robust in its ability to translate and scale. Differing patterns, including groupings of cells of various sizes and arranged in variously sized cell blocks, can be used. For example, 4×4 cell blocks of 8×8 pixel cells with an overlap of half of the block can be used. Histograms of channels can be used, including nine channels or bins evenly spread over 0-180 degrees. In this example, the HoG descriptor on a 96×96 image is 25 blocks×16 cells×9 bins=3600, the latter quantity representing the dimension. AU occurrences can be rendered. The videos can be grouped into demographic datasets based on nationality and/or other demographic parameters for further detailed analysis.

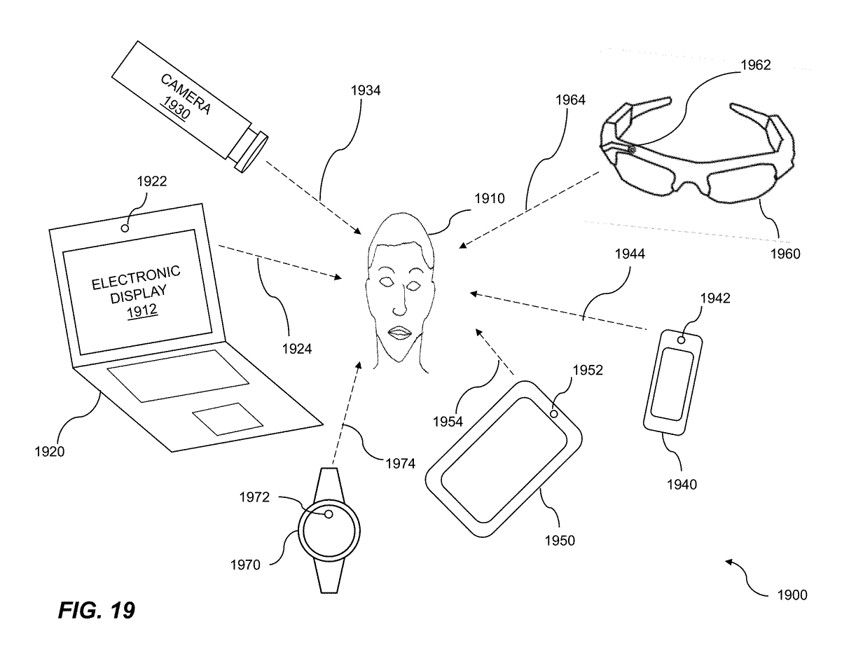

FIG. 19 shows example image collection including multiple mobile devices 1900. The images that can be collected can be analyzed to perform mental state analysis as well as to determine weights and image classifiers. The weights and the image classifiers can be used to infer an emotional metric. The multiple mobile devices can be used to collect video data on a person. While one person is shown, in practice the video data on any number of people can be collected. A user 1910 can be observed as she or he is performing a task, experiencing an event, viewing a media presentation, and so on. The user 1910 can be viewing a media presentation or another form of displayed media. The one or more video presentations can be visible to a plurality of people instead of an individual user. If the plurality of people is viewing a media presentation, then the media presentations can be displayed on an electronic display 1912. The data collected on the user 1910 or on a plurality of users can be in the form of one or more videos. The plurality of videos can be of people who are experiencing different situations. Some example situations can include the user or plurality of users viewing one or more robots performing various tasks. The situations could also include exposure to media such as advertisements, political messages, news programs, and so on. As noted before, video data can be collected on one or more users in substantially identical or different situations. The data collected on the user 1910 can be analyzed and viewed for a variety of purposes, including expression analysis. The electronic display 1912 can be on a laptop computer 1920 as shown, a tablet computer 1950, a cell phone 1940, a television, a mobile monitor, or any other type of electronic device. In a certain embodiment, expression data is collected on a mobile device such as a cell phone 1940, a tablet computer 1950, a laptop computer 1920, or a watch 1970. Thus, the multiple sources can include at least one mobile device such as a cell phone 1940 or a tablet computer 1950, or a wearable device such as a watch 1970 or glasses 1960. A mobile device can include a front-side camera and/or a back-side camera that can be used to collect expression data. Sources of expression data can include a webcam 1922, a smartphone camera 1942, a tablet camera 1952, a wearable camera 1962, and a mobile camera 1930. A wearable camera can comprise various camera devices such as the watch camera 1972.

As the user 1910 is monitored, the user 1910 might move due to the nature of the task, boredom, discomfort, distractions, or for another reason. As the user moves, the camera with a view of the user's face can change. Thus, as an example, if the user 1910 is looking in a first direction, the line of sight 1924 from the webcam 1922 is able to observe the individual's face, but if the user is looking in a second direction, the line of sight 1934 from the mobile camera 1930 is able to observe the individual's face. Further, in other embodiments, if the user is looking in a third direction, the line of sight 1944 from the phone camera 1942 is able to observe the individual's face, and if the user is looking in a fourth direction, the line of sight 1954 from the tablet camera 1952 is able to observe the individual's face. If the user is looking in a fifth direction, the line of sight 1964 from the wearable camera 1962, which can be a device such as the glasses 1960 shown and can be worn by another user or an observer, is able to observe the individual's face. If the user is looking in a sixth direction, the line of sight 1974 from the wearable watch-type device 1970 with a camera 1972 included on the device, is able to observe the individual's face. In other embodiments, the wearable device is another device, such as an earpiece with a camera, a helmet or hat with a camera, a clip-on camera attached to clothing, or any other type of wearable device with a camera or another sensor for collecting expression data. The user 1910 can also employ a wearable device including a camera for gathering contextual information and/or collecting expression data on other users. Because the user 1910 can move her or his head, the facial data can be collected intermittently when the individual is looking in a direction of a camera. In some cases, multiple people are included in the view from one or more cameras, and some embodiments include filtering out faces of one or more other people to determine whether the user 1910 is looking toward a camera. All or some of the expression data can be continuously or sporadically available from these various devices and other devices.

The captured video data can include facial expressions and can be analyzed on a computing device, such as the video capture device or on another separate device. The analysis of the video data can include the use of a classifier. For example, the video data can be captured using one of the mobile devices discussed above and sent to a server or another computing device for analysis. However, the captured video data including expressions can also be analyzed on the device which performed the capturing. For example, the analysis can be performed on a mobile device, where the videos were obtained with the mobile device and wherein the mobile device includes one or more of a laptop computer, a tablet, a PDA, a smartphone, a wearable device, and so on. In another embodiment, the analyzing comprises using a classifier on a server or other computing device other than the capturing device. The result of the analyzing can be used to infer one or more emotional metrics. In embodiments, the plurality of sources of facial data includes one or more of a webcam, a smartphone camera, a tablet camera, an automobile camera, a connected home camera, a social robot, a wearable camera, or a wearable camera comprising glasses worn by an observer.

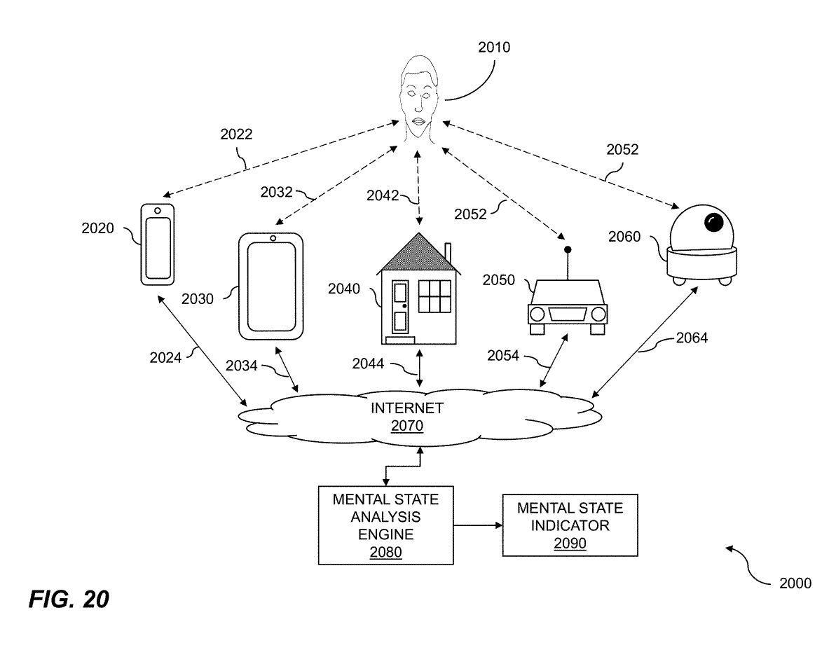

FIG. 20 illustrates mental state analysis using one or more connected, computer-based devices. In illustration 2000, an individual 2010 can interact with many different kinds of computer-based devices 2020, 2030, 2040, 2050, and 2060. The various computer-based devices can be used to obtain facial data of the individual 2010. The facial data can be used in collecting mental state data. In embodiments, one or more selected portions of mental state data tagged with additional data can be analyzed to generate mental state information. For example, mobile phone 2020 can have line of sight 2022 from one or more integrated cameras to individual 2010 for purposes of obtaining video of the individual; tablet 2030 can have line of sight 2032 from one or more integrated cameras to individual 2010 for purposes of obtaining video of the individual; smart house 2040 can have line of sight 2042 from one or more integrated cameras to individual 2010 for purposes of obtaining video of the individual; smart automobile 2050 can have line of sight 2052 from one or more integrated cameras to individual 2010 for purposes of obtaining video of the individual; social robot 2060 can have line of sight 2052 from one or more integrated cameras to individual 2010 for purposes of obtaining video of the individual. The integrated cameras of computer-based devices 2020, 2030, 2040, 2050, and 2060 can be webcams. As the term is used herein, webcams can refer to a camera on a computer (such as a laptop, a net-book, a tablet, a wearable device, or the like), a video camera, a still camera, a cell phone camera, a camera mounted in a transportation vehicle, a wearable device including a camera, a mobile device camera (including, but not limited to, a front side camera), a thermal imager, a CCD device, a three-dimensional camera, a depth camera, a social robot camera, multiple webcams used to capture different views of individuals, or any other type of image capture apparatus that allows image data to be captured and used by an electronic system.

Illustration 2000 includes a computing network, such as the Internet 2070, connected to the various computer-based devices. For example, device 2020 is connected to the Internet 2070 over network link 2024; device 2030 is connected to the Internet 2070 over network link 2034; device 2040 is connected to the Internet 2070 over network link 2044; device 2050 is connected to the Internet 2070 over network link 2054; device 2060 is connected to the Internet 2070 over network link 2064. The network link can be a wireless link, a wired link, and so on. Through a network, such as the Internet 2070, a mental state analysis engine 2080 can receive the video obtain by computer-based devices 2020, 2030, 2040, 2050, and 2060 for processing and analysis. In embodiments, illustration 2000 shows a computer-implemented method for mental state analysis. The analysis can generate mental state information from one or more selected portions of mental state data. The analysis can include: receiving two or more portions of collected mental state data tagged with additional information, wherein the two or more portions of mental state data come from a plurality of sources of facial data, wherein the mental state data collected is intermittent, and wherein the plurality of sources includes at least one computer-based device; interpolating the intermittent mental state data, wherein the interpolating is based on the additional information that was tagged; selecting one or more portions of the received two or more portions of mental state data based on the additional information that was tagged, wherein the one or more selected portions of mental state data are selected based, at least in part, on tags identifying a particular context; and analyzing, using one or more processors of mental state analysis engine 2080, the one or more selected portions of mental state data to generate mental state information. In embodiments, a result from the analyzing can be a mood measurement, illustrated by mental state indicator 2090.

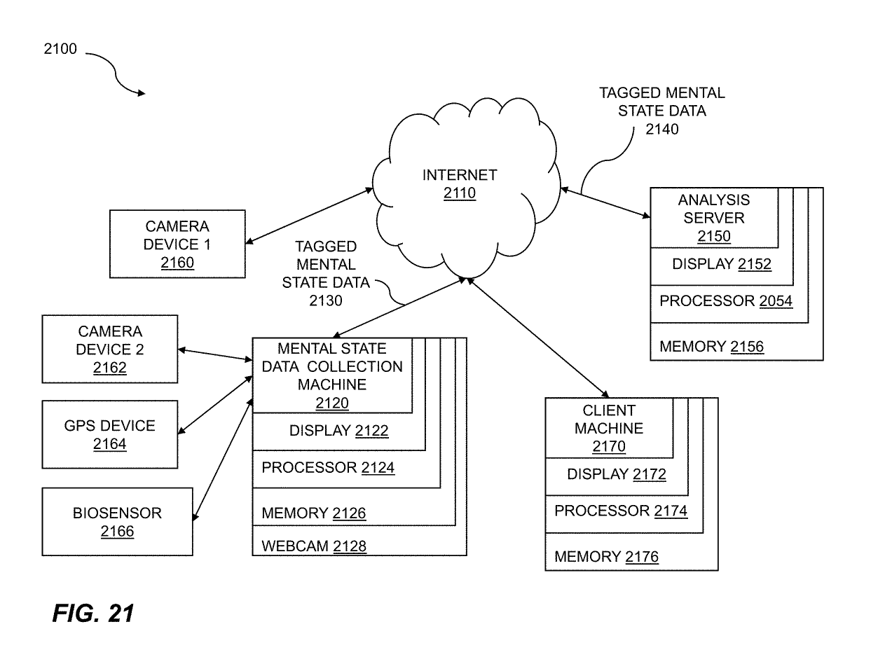

FIG. 21 is a system diagram for mental state analysis. The system 2100 can include one or more computers coupled together by a communication link such as the Internet 2110 and can be used for a computer-implemented method for mental state analysis. The system 2100 can also include two or more cameras that can be linked to the one or more computers and/or directly to a communication link. The system 2100 can include a mental state data collection machine 2120, which is referred to as a client machine in some embodiments. The mental state data collection machine 2120 includes a memory 2126 which stores instructions, one or more processors 2124 coupled to the memory, a display 2122, and, in some embodiments, a webcam 2128. The display 2122 can be any electronic display, including but not limited to, a computer display, a laptop screen, a net-book screen, a tablet screen, a cell phone display, a mobile device display, a remote with a display, a television, a projector, or the like. The webcam 2128, as the term is used herein, can refer to a camera on a computer (such as a laptop, a net-book, a tablet, or the like), a video camera, a still camera, a cell phone camera, a mobile device camera (including, but not limited to, a front-side camera), a thermal imager, a CCD device, a three-dimensional camera, a depth camera, multiple webcams used to capture different views of viewers, or any other type of image capture apparatus that allows image data captured to be used by an electronic system. In some embodiments, a second camera device 2162, a GPS device 2164, and/or a biosensor 2166 can be coupled to the mental state data collection machine 2120. The second camera device 2162 and/or the webcam 2128 can be used to capture facial images of an individual that can be used as mental state data. Likewise, the biosensor 2166 can capture mental state data from the individual. The GPS device 2164 can be used to obtain location data which can then be used to provide contextual information about the mental state data. Other sensors or programs running on the mental state data collection machine can be used to gather additional data relating to the mental state data.

The individual can interact with the mental state data collection machine 2120, interact with another computer, view a media presentation on another electronic display, and/or perform numerous other activities. The system 2100 can include a computer program product embodied in a non-transitory computer readable medium for mental state analysis, the computer program product comprising code which causes one or more processors to perform operations of: capturing mental state data on an individual from a first source that includes facial information, wherein the mental state data collected is intermittent; capturing mental state data on the individual from at least a second source that includes facial data, wherein the at least a second source comprises a computer-based device; determining additional data about the mental state data wherein the additional data provides information about mental states and wherein the additional data includes information about a context as the mental state data was collected; tagging the additional data to the mental state data; interpolating the intermittent mental state data, wherein the interpolating is based on the additional data that was tagged; and sending at least a portion of the mental state data tagged with the additional data 2130 to a web service. With such a program stored in memory, the one or more processors 2124 can be configured to capture mental state data on an individual from a first source, determine additional data about the mental state data wherein the additional data provides information about mental states, tag the additional data to the mental state data, and send to a web service at least a portion of the mental state data tagged with additional data 2130. In some embodiments, the second camera device 2162 can be used as a second source of mental state data which is tagged with the additional data and sent to the web service. In some embodiments, the captured mental state data is intermittent or missing and is interpolated and/or imputed. The interpolation can be performed on the mental state data collection machine 2120, the analysis server 2150 described forthwith, or another computer not shown.

Some embodiments can include an analysis server 2150. In embodiments, the analysis server 2150 can be configured as a web service. The analysis server 2150 includes one or more processors 2154 coupled to a memory 2156 to store instructions. Some embodiments of the analysis server 2150 include a display 2152. The one or more processors 2154 can be configured to receive tagged mental state data 2140 from the mental state data collection machine 2120, the first camera device 2160, and/or any other computers configured to collect mental state data. The one or more processors 2154 can then select one or more portions of the received mental state data 2140 based on the additional data from the tags, and can then analyze the received mental state data 2140. The analysis can produce mental state information, inferred mental states, emotigraphs, actigraphs, other textual/graphical representations, mood measurements, or any other type of analysis. The analysis server 2150 can display at least some of the analysis on the display 2152 and/or can provide the analysis of the mental state data to a client machine, such as the mental state data collection machine 2120, or another client machine 2170, so that the analysis can be displayed to a user. The analysis server 2150 can enable a method that includes receiving two or more portions of collected mental state data tagged with additional information, wherein the two or more portions of mental state data come from a plurality of sources of facial data, wherein the mental state data collected is intermittent, and wherein the plurality of sources includes at least one computer-based device; interpolating the intermittent mental state data, wherein the interpolating is based on the additional information that was tagged; selecting one or more portions of the received two or more portions of mental state data based on the additional information that was tagged, wherein the one or more selected portions of mental state data are selected based, at least in part, on tags identifying a particular context; and analyzing, using one or more processors, the one or more selected portions of mental state data to generate mental state information.

Some embodiments include another client machine 2170. The client machine includes one or more processors 2174 coupled to memory 2176 to store instructions, and a display 2172. The client machine can receive the analysis of the mental state data from the analysis server 2150 and can render an output to the display 2172. The system 2100 can enable a computer-implemented method for mental state analysis that includes receiving an analysis based on both mental state data and additional data tagged to the mental state data, and rendering an output based on the analysis. In at least one embodiment the mental state data collection machine, the analysis server, and/or the client machine functions are accomplished by one computer.

Thus, the system 2100 can enable a method for mental state analysis comprising: capturing mental state data on an individual from a first source that includes facial information, wherein the mental state data collected is intermittent; capturing mental state data on the individual from at least a second source that includes facial data, wherein the at least a second source comprises a computer-based device; determining additional data about the mental state data wherein the additional data provides information about mental states and wherein the additional data includes information about a context as the mental state data was collected; tagging the additional data to the mental state data; interpolating the intermittent mental state data, wherein the interpolating is based on the additional data that was tagged; and sending at least a portion of the mental state data tagged with the additional data to a web service.

Each of the above methods may be executed on one or more processors on one or more computer systems. Embodiments may include various forms of distributed computing, client/server computing, and cloud based computing. Further, it will be understood that the depicted steps or boxes contained in this disclosure's flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. The elements and combinations of elements in the block diagrams and flow diagrams, show functions, steps, or groups of steps of the methods, apparatus, systems, computer program products and/or computer-implemented methods. Any and all such functions—generally referred to herein as a “circuit,” “module,” or “system”—may be implemented by computer program instructions, by special-purpose hardware-based computer systems, by combinations of special purpose hardware and computer instructions, by combinations of general purpose hardware and computer instructions, and so on.

A programmable apparatus which executes any of the above mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are neither limited to conventional computer applications nor the programmable apparatus that run them. To illustrate: the embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM), an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the causal entity.

While the invention has been disclosed in connection with preferred embodiments shown and described in detail, various modifications and improvements thereon will become apparent to those skilled in the art. Accordingly, the forgoing examples should not limit the spirit and scope of the present invention; rather it should be understood in the broadest sense allowable by law.

What is claimed is:

1. A computer-implemented method for mental state analysis comprising:
receiving two or more portions of collected mental state data tagged with additional information, wherein the two or more portions of mental state data come from a plurality of sources of facial data, wherein the mental state data collected is intermittent, and wherein the plurality of sources includes at least one computer-based device;
interpolating the intermittent mental state data, wherein the interpolating is based on the additional information that was tagged;
selecting one or more portions of the received two or more portions of mental state data based on the additional information that was tagged, wherein the one or more selected portions of mental state data are selected based, at least in part, on tags identifying a particular context; and
analyzing, using one or more processors, the one or more selected portions of mental state data to generate mental state information.

2. The method of claim 1 wherein a result from the analyzing is a mood measurement.

3. The method of claim 1 wherein the plurality of sources of facial data includes one or more of a webcam, a phone camera, a tablet camera, an automobile camera, a connected home camera, a social robot, a wearable camera, or a wearable camera comprising glasses worn by an observer.

4. The method of claim 1 wherein the one or more selected portions of mental state data are selected based, at least in part, on tags identifying a particular individual.

5. The method of claim 4 wherein the one or more selected portions of mental state data are selected based, at least in part, on tags identifying one or more contexts.

6. The method of claim 4 wherein the one or more selected portions of mental state data are selected to include tags identifying at least two different timestamps.

7. The method of claim 1 further comprising sending output for rendering to another computer, wherein the other computer provides the rendering.

8. The method of claim 1 further comprising imputing additional mental state data where the mental state data is missing.

9. The method of claim 1 further comprising associating interpolated data with the mental state data that is collected on an intermittent basis.

10. The method of claim 1 wherein the mental state data is intermittent due to an image collection being lost.

11. The method of claim 1 wherein mental state data is collected from multiple devices while a user is performing a task using an electronic display during a portion of time.

12. The method of claim 11 wherein the multiple devices include a tablet computer or a cell phone.

13. The method of claim 1 wherein contextual data is collected simultaneously with the mental state data.

14. (canceled)

15. The method of claim 1 further comprising evaluating a temporal signature for the mental states.

16. The method of claim 15 further comprising using the temporal signature to infer additional mental states.

17. The method of claim 1 wherein the analyzing mental state data to produce mental state information further comprises analyzing an emotional mood associated with the mental state information.

18. The method of claim 17 wherein the analyzing the emotional mood is used to provide emotional health tracking.

19. A computer-implemented method for mental state analysis comprising:
capturing mental state data on an individual from a first source that includes facial information, wherein the mental state data collected is intermittent;
capturing mental state data on the individual from at least a second source that includes facial data, wherein the at least a second source comprises a computer-based device;
determining additional data about the mental state data wherein the additional data provides information about mental states and wherein the additional data includes information about a context as the mental state data was collected;
tagging the additional data to the mental state data;
interpolating the intermittent mental state data, wherein the interpolating is based on the additional data that was tagged; and
sending at least a portion of the mental state data tagged with the additional data to a web service.

20. The method of claim 19 further comprising locating pertinent mental state data based on the tagging.

21. (canceled)

22. The method of claim 20 further comprising sending tagged mental state data over the internet to cloud or web-based storage or web-based services for remote use and using the tags locally on a machine where the mental state data was collected.

23. The method of claim 19 further comprising analyzing the mental state data to produce mental state information.

24. The method of claim 23, further comprising using the additional data in conjunction with the mental state data to produce the mental state information.

25. The method of claim 19 further comprising obtaining mental state data from a second source.

26. The method of claim 25 wherein the mental state data from the second source includes facial information.

27. The method of claim 26 wherein the mental state data from the second source includes biosensor information.

28-38. (canceled)

39. The method of claim 19 further comprising performing unsupervised learning, wherein the unsupervised learning enables the interpolating based on the additional data that was tagged.

40. The method of claim 39 wherein the unsupervised learning further comprises learning additional data about the mental state data, wherein the learning is based on mental state information and mental state information collection context.

41. A computer program product embodied in a non-transitory computer readable medium for mental state analysis, the computer program product comprising code which causes one or more processors to perform operations of:
capturing mental state data on an individual from a first source that includes facial information, wherein the mental state data collected is intermittent;
capturing mental state data on the individual from at least a second source that includes facial data, wherein the at least a second source comprises a computer-based device;
determining additional data about the mental state data wherein the additional data provides information about mental states and wherein the additional data includes information about a context as the mental state data was collected;
tagging the additional data to the mental state data;
interpolating the intermittent mental state data, wherein the interpolating is based on the additional data that was tagged; and
sending at least a portion of the mental state data tagged with the additional data to a web service.
